(19) United States
(12) Patent Application Publication
(10) Pub. No.: US 2013/0171122 A1
(43) Pub. Date: Jul. 4, 2013

(54) ENDOPEPTIDASE AND NEUROTOXIN COMBINATION TREATMENT OF BLADDER DISORDERS

(71) Applicant: Allergan, Inc., Irvine, CA (US)

(72) Inventors: Gregory F. Brooks, Irvine, CA (US); Kenton B. Abel, Hacienda Heights, CA (US)

(73) Assignee: ALLERGAN, INC., Irvine, CA (US)

(21) Appl. No.: 13/726,801

(22) Filed: Dec. 26, 2012

Related U.S. Application Data
(60) Provisional application No. 61/581,590, filed on Dec. 29, 2011.

(51) Int. Cl. A61K 38/48

(52) U.S. Cl. CPC A61K 38/4893; A61K 38/4886
USPC 424/94.3

(57) ABSTRACT

The present specification discloses Clostridial neurotoxins and TEMs, compositions comprising such Clostridial neurotoxins and TEMs, kits comprising such Clostridial neurotoxins, TEMs and/or compositions, methods of treating a bladder disorder in an individual using such Clostridial neurotoxins, TEMs and/or compositions, use of such Clostridial neurotoxins and TEMs in manufacturing a medicament for treating a bladder disorder, and uses of such Clostridial neurotoxins, TEMs and/or compositions in treating a bladder disorder.

The application claims priority pursuant to 35 U.S.C. §119(e) to U.S. Provisional Application 61/581,590 filed on Dec. 29, 2011, incorporated entirely by reference.

### BACKGROUND

Urine is made in the kidneys, and travels down two tubes called ureters to the urinary bladder. Located just above and behind the pubic symphysis in the anterior pelvis, the bladder serves solely as a reservoir, storing urine before disposal through the urethra by urination. The ability of the body to store urine allows urination to be infrequent and voluntary. The normal capacity of the bladder is about 300 mL to about 600 mL. As urine accumulates, the bladder thins as it stretches, allowing the bladder to store larger amounts of urine without a significant rise in internal pressure. During urination, the bladder muscles contract, and two sphincters (valves) open to allow urine to flow out. Urine exits the bladder into the urethra, which carries urine out of the body. Because it passes through the penis, the urethra is longer in men (8 inches) than in women (1.5 inches).

An extraperitoneal musculomembranous organ having a lumen, anatomically the bladder is essentially a sac comprising four major layers. The outer layer is called the tunica serosa and is a partial layer derived from the peritoneum. The tunica muscularis is next layer, and is made up of primarily of the detrusor muscle of the urinary bladder wall. The detrusor muscle includes three smooth muscle fibers arranged in spiral, longitudinal, and circular bundles. This configuration allows the bladder to stretch in order to accommodate urine inflow and, upon contraction, decrease bladder size in all dimensions to enable effective and complete expeltion of the urine. The next layer tela submucosa is a thin layer of loose connective tissue between the tunica muscularis with the tunica mucosa. The loose texture of the tela submucosa layer allows the tunica mucosa to be thrown into folds (called rugae) when the bladder is empty. The tunica mucosa is the innermost layer and contains two layers, the lamina propia and transitional epithelium. When empty, the urinary bladder collapses and the tunica mucosa develops rugae. As it fills with urine, the folds become distended and the tunica mucosa becomes smooth.

Although its size, position, and relations vary according to the amount of urine, the bladder contains several features useful as landmarks. A triangle-shaped organ, the bladder is positioned so that the base is located dorsally and the vertex is ventrally. The anterior or superior surface region forming the roof of the bladder is called the dome. The dome region of the bladder is covered by peritoneum and the median umbilical ligament anchors the bladder to the umbilicus. The regions forming the three bladder sides can be divided into a left inferolateral bladder wall region, right inferolateral bladder wall region, and posterior bladder wall region. The anterior angle or apex is formed by the convergence of the left and right inferolateral bladder wall regions. The apex is attached to the anterior abdominal wall by the urachus, a ligament that extends superiorly to the umbilicus as the median umbilical ligament. The ureters attach to the bladder at the left and right posterolateral angles. The posterolateral angles also mark where the dome region, posterior bladder wall and left or right inferolateral bladder wall regions come together.

The posterior region of the bladder is called the neck (or base) and includes the inferior angle, the point where the urethra leaves the neck, and the internal urethral sphincter, an involuntary smooth muscle. At the bladder neck, the tunica muscularis is more organized, and three relatively distinct layers become apparent. The inner longitudinal layer fuses with the inner longitudinal layer of the urethra. The middle circular layer is most prominent in the proximity of the bladder neck, and it fuses with the deep trigonal muscle. The outer longitudinal layer contributes some anterior fibers to what becomes the pubovesical muscle, terminating on the posterior surface of the pubic bone. These muscles appear to be important in opening the bladder neck during micturition. Posteriorly, the outer longitudinal fibers interdigitate with deep trigonal fibers and the detrusor muscle. These fibers may aid in bladder neck closure. In males, the bladder neck is contiguous with the prostate, which is attached to the pubis by puboprostatic ligaments. In females, pubourethral ligaments support the bladder neck and urethra. Posterior to the bladder neck, the urethra is encircled by the external urethral sphincter, a voluntary muscle.

The features observable on the inside of the bladder are the ureter orifices, the trigone, and the internal orifice of the urethra. The trigone is located on the internal face of the posterior wall of the bladder. A triangular region, the boarders of the trigone are defined by the internal urethral orifice and the orifices of the right and left ureter. The trigone region is always smooth in appears because the tunica mucosa is firmly attached to the tunica muscularis. Thus, rugae are never present in the trigone even when the bladder is empty. The superior border of the trigone is a raised area called the interureteric ridge. Deep to the tunica mucosa are two muscular layers. The superficial layer connects to longitudinal urethral musculature. The deep muscle fuses with detrusor and Waldeyer sheath, the fibromuscular covering of the intramural ureter. The intramural ureter enters the bladder wall obliquely. The muscle fibers are longitudinal in orientation at this point. This segment of the ureter is about 1.5 cm in length.

The bladder receives motor innervation from both sympathetic efferent nerves, most of which arise from the hypogastric plexuses and nerves from the lumbar spinal cord, and parasympathetic efferent nerves, which come from the pelvic splanchnic nerves and the inferior hypogastric plexus. Sensation from the bladder is transmitted to the central nervous system (CNS) via general visceral afferent (GVA) nerves. GVA nerves on the superior dome surface follow the course of the sympathetic efferent nerves back to the CNS, while GVA nerves on the inferior neck portion of the bladder follow the course of the parasympathetic efferent nerves. Interneurons from the CNS make connections to both the sympathetic efferent nerves and parasympathetic efferent nerves. Somatic efferent nerves from the CNS innervate the external urethral sphincter.

A normal bladder functions through a complex coordination of musculoskeletal, neurologic, and psychological functions that allow filling and emptying of the bladder contents. A micturition response occurs when urine accumulation expands the bladder beyond a threshold point, typically when the volume of urine reaches about 400 mL. This distension triggers stretch receptors located at the dome of the bladder to stimulate sympathetic efferent nerves to send signals to the pons which activate the pontine micturition center located in the rostral pons in the brainstem. This activation in turn initiates a response via interneurons which 1) signals parasympathetic efferent nerves to trigger contraction of the detrusor muscle and relaxation of the internal urethral sphincter; and 2) signals somatic efferent nerves to trigger relaxation of the external urethral sphincter. This encourages the bladder to expel urine through the urethra. A micturition response can also be elicited through the conscious desire to void.

During urinary continence, urine accumulation is not yet reached a point where the bladder is stretched enough to initiate a micturition response. Neurons within the pontine micturition center are not stimulated and continence is achieved by the synergic relaxation of detrusor muscles and contraction of the bladder neck, internal urethral sphincter, and pelvic floor muscles. Urination can be prevented by cortical suppression of the peripheral nervous system or by voluntary contraction of the external urethral sphincter. In summary, the normal function of the urinary bladder requires regulatory input from motor, sympathetic, parasympathetic, and/or sensory neurons.

Clostridial toxins inhibit motor neurons by blocking the release of acetylcholine (ACh) at the pre-synaptic neuromuscular junction. The ability of Clostridial toxins, such as, e.g., Botulinum neurotoxins (BoNTs), Botulinum neurotoxin serotype A (BoNT/A), Botulinum neurotoxin serotype B (BoNT/B), Botulinum neurotoxin serotype C1 (BoNT/C1), Botulinum neurotoxin serotype D (BoNT/D), Botulinum neurotoxin serotype E (BoNT/E), Botulinum neurotoxin serotype F (BoNT/F), and Botulinum neurotoxin serotype G (BoNT/G), and Tetanus neurotoxin (TeNT), to inhibit neuronal transmission are being exploited in a wide variety of therapeutic and cosmetic applications. As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder.

Generally, administration of a Clostridial toxin treatment is well tolerated. However, toxin administration in some applications can be challenging because of the larger doses required to achieve a beneficial effect. Larger doses can increase the likelihood that the toxin may move through the interstitial fluids and the circulatory systems, such as, e.g., the cardiovascular system and the lymphatic system, of the body, resulting in the undesirable dispersal of the toxin to areas not targeted for toxin treatment. Such dispersal can lead to undesirable side effects, such as, e.g., inhibition of neurotransmitter release in neurons not targeted for treatment or paralysis of a muscle not targeted for treatment. For example, a individual administered a therapeutically effective amount of a BoNT/A treatment into the bladder for overactive bladder may develop dry mouth and/or dry eyes. Thus, there still remains a need for treatments having the therapeutic effects that only larger doses of a Clostridial toxin can currently provide, but reduce or prevent the undesirable side-effects associated with larger doses of a Clostridial toxin administration.

A Clostridial toxin treatment inhibits neurotransmitter release by disrupting the exocytotic process used to secret the neurotransmitter into the synaptic cleft. There is a great desire by the pharmaceutical industry to expand the use of Clostridial toxin therapies beyond its current myo-relaxant applications to treat sensory, sympathetic, and/or parasympathetic nerve-based ailments, such as, e.g., various kinds of involuntary movement disorders. One approach that is currently being exploited involves modifying a Clostridial toxin such that the modified toxin has an altered cell targeting capability for a neuronal or non-neuronal cell of interest. Called re-targeted endopeptidases or Targeted Vesicular Exocytosis Modulator Proteins (TVEMPs) or Targeted Exocytosis Modulators (TEMs), these molecules achieve their exocytosis inhibitory effects by targeting a receptor present on the neuronal or non-neuronal target cell of interest. This re-targeted capability is achieved by replacing the naturally-occurring binding domain of a Clostridial toxin with a targeting domain showing a selective binding activity for a non-Clostridial toxin receptor present in a cell of interest. Such modifications to the binding domain result in a molecule that is able to selectively bind to a non-Clostridial toxin receptor present on the target cell. A re-targeted endopeptidase can bind to a target receptor, translocate into the cytoplasm, and exert its proteolytic effect on the SNARE complex of the neuronal or non-neuronal target cell of interest.

The present specification discloses methods for treating an individual suffering from a bladder disorder. This is accomplished by employing a combined therapy that comprises administering a therapeutically effective amount of a composition comprising a Clostridial toxin as well as administering a therapeutically effective amount of a composition comprising a TEM to an individual in need thereof. The disclosed methods provide a safe, inexpensive, out patient-based treatment for the treatment of bladder disorders. In addition, the therapies disclosed herein reduce or prevent unwanted side-effects associated with larger Clostridial toxin doses. These and related advantages are useful for various clinical applications, such as, e.g., the treatment of bladder disorders where a larger amount of a Clostridial toxin to an individual could produce a beneficial effect, but for the undesirable side-effects.

### SUMMARY

With reference to bladder disorders as disclosed herein, and without wishing to be limited by any particular theory, it is believed that motor, sympathetic, parasympathetic, and/or sensory neurons have important functions in aspects of bladder disorders and that improper innervations from these types of neurons can contribute to one or more different types of bladder disorders. It is further theorized that such a TEM in combination with a Clostridial toxin can provide enhanced, if not synergistic, therapeutic benefit because such a combination also inhibit motor neurons. However, using a combination therapy of such a TEM with a Clostridial toxin, also may allow a lower dose of a Clostridial toxin to be administered to treat a bladder disorder. This will result in a decrease in muscle weakness generated in the compensatory muscles relative to the current treatment paradigm. As such, a combined therapy using a Clostridial toxin and a TEM comprising a targeting domain for a receptor present on sympathetic, parasympathetic, and/or sensory neurons can reduce or prevent these improper innervations, and in combination can reduce or prevent one or more symptoms associate with a bladder disorder.

Thus, aspects of the present specification disclose methods of treating a bladder disorder in an individual, the methods comprising the step of administering to the individual in need thereof a therapeutically effective amount of a composition including a Clostridial neurotoxin and a TEM, wherein administration of the composition reduces a symptom of the bladder disorder, thereby treating the individual. A Clostridial neurotoxin includes, without limitation, a Botulinum toxin (BoNT), a Tetanus toxin (TeNT), a Baratii toxin (BaNT), and a Butyricum toxin (BuNT). In some aspects, a TEM may comprise a targeting domain, a Clostridial toxin translocation domain and a Clostridial toxin enzymatic domain. In some aspects, a TEM may comprise a targeting domain, a Clostridial toxin translocation domain, a Clostridial toxin enzymatic domain, and an exogenous protease cleavage site. A targeting domain includes, without limitation, a sensory neuron targeting domain, a sympathetic neuron targeting domain, or a parasympathetic neuron targeting domain. A bladder disorder includes, without limitation, urinary incontinence, overactive bladder, detrusor dysfunction, lower urinary tract dysfunction, urinary retention, urinary hesitancy, polyuria, nocturia, chronic urinary tract infection, a bladder disorder associated with a prostate disorder, and a bladder disorder associated with a neurogenic dysfunction (such as, e.g., Parkinson's Disease, multiple sclerosis, spina bifida, transverse myelitis, stroke, spinal cord injury, spasm reflex, and a neurologic lesion of the spinal cord or brain).

Other aspects of the present specification disclose uses of a Clostridial neurotoxin and a TEM disclosed herein in the manufacturing a medicament for treating a bladder disorder disclosed herein in an individual in need thereof.

Yet other aspects of the present specification uses of a Clostridial neurotoxin in conjunction with a TEM in the treatment of a bladder disorder in an individual in need thereof. Additional aspects include uses of a composition including a Clostridial neurotoxin in conjunction with a composition including a TEM in the treatment of a bladder disorder in an individual in need thereof.

Still other aspects of the present specification disclose kits useful for treating a bladder disorder. A kit can comprise a Clostridial neurotoxin disclosed herein or a composition including such a Clostridial neurotoxin and a TEM disclosed herein or a composition including such a TEM disclosed herein. A kit can further comprise instructions on how to administer the Clostridial neurotoxin and the TEM in a combined therapy for the treatment of a bladder disorder.

### BRIEF DESCRIPTION OF THE DRAWINGS

FIG. 1 shows a schematic of a bladder with one possible injection paradigm.

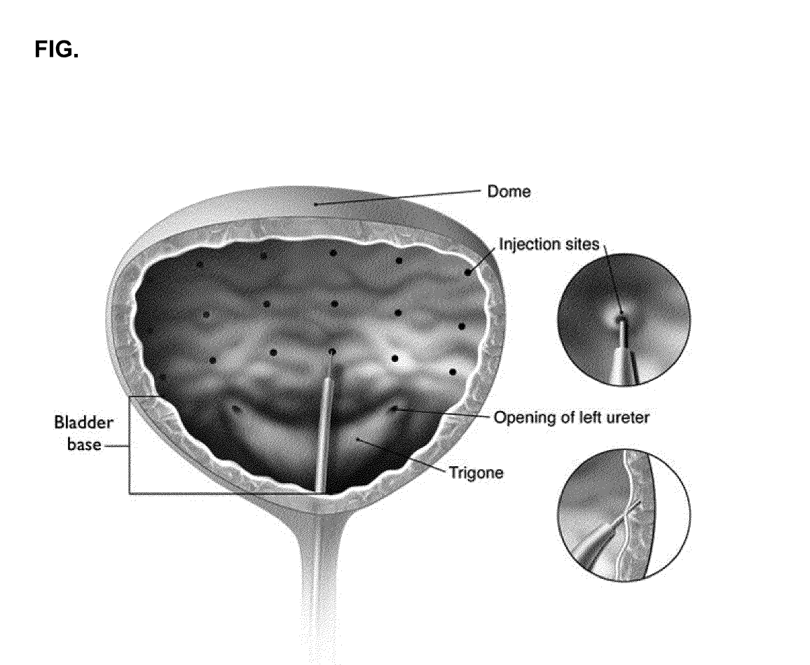

### DESCRIPTION

Clostridia toxins produced by Clostridium botulinum, Clostridium tetani, Clostridium baratii and Clostridium butyricum are the most widely used in therapeutic and cosmetic treatments of humans and other mammals. Strains of C. botulinum produce seven antigenically-distinct types of Botulinum toxins (BoNTs), which have been identified by investigating botulism outbreaks in man (BoNT/A, BoNT/B, BoNT/E and BoNT/F), animals (BoNT/C1 and BoNT/D), or isolated from soil (BoNT/G). BoNTs possess approximately 35% amino acid identity with each other and share the same functional domain organization and overall structural architecture. It is recognized by those of skill in the art that within each type of Clostridial toxin there can be subtypes that differ somewhat in their amino acid sequence, and also in the nucleic acids encoding these proteins. For example, there are presently five BoNT/A subtypes, BoNT/A1, BoNT/A2, BoNT/A3 BoNT/A4 and BoNT/A5, with specific subtypes showing approximately 89% amino acid identity when compared to another BoNT/A subtype. While all seven BoNT serotypes have similar structure and pharmacological properties, each also displays heterogeneous bacteriological characteristics. In contrast, tetanus toxin (TeNT) is produced by a uniform group of C. tetani. Two other Clostridia species, C. baratii and C. butyricum, produce toxins, BaNT and BuNT, which are functionally similar to BoNT/F and BoNT/E, respectively.

Clostridial toxins are released by Clostridial bacterium as complexes comprising the approximately 150-kDa Clostridial toxin along with associated non-toxin proteins (NAPs). Identified NAPs include proteins possessing hemaglutination activity, such, e.g., a hemagglutinin of approximately 17-kDa (HA-17), a hemagglutinin of approximately 33-kDa (HA-33) and a hemagglutinin of approximately 70-kDa (HA-70); as well as non-toxic non-hemagglutinin (NTNH), a protein of approximately 130-kDa. Thus, the botulinum toxin type A complex can be produced by Clostridial bacterium as 900-kDa, 500-kDa and 300-kDa forms. Botulinum toxin types B and C₁ are apparently produced as only a 500-kDa complex. Botulinum toxin type D is produced as both 300-kDa and 500-kDa complexes. Finally, botulinum toxin types E and F are produced as only approximately 300-kDa complexes. The differences in molecular weight for the complexes are due to differing ratios of NAPs. The toxin complex is important for the intoxication process because it provides protection from adverse environmental conditions, resistance to protease digestion, and appears to facilitate internalization and activation of the toxin.

A Clostridial toxin itself is translated as a single chain polypeptide that is subsequently cleaved by proteolytic scission within a disulfide loop by a naturally-occurring protease. This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains. The naturally-occurring protease used to convert the single chain molecule into the di-chain is currently not known. In some serotypes, such as, e.g., BoNT/A, the naturally-occurring protease is produced endogenously by the bacteria serotype and cleavage occurs within the cell before the toxin is release into the environment. However, in other serotypes, such as, e.g., BoNT/E, the bacterial strain appears not to produce an endogenous protease capable of converting the single chain form of the toxin into the di-chain form. In these situations, the toxin is released from the cell as a single-chain toxin which is subsequently converted into the di-chain form by a naturally-occurring protease found in the environment.

Each mature di-chain molecule of a Clostridial toxin comprises three functionally distinct domains: 1) an enzymatic domain located in the light chain (LC) that includes a metalloprotease region containing a zinc-dependent endopeptidase activity which specifically targets core components of the neurotransmitter release apparatus; 2) a translocation domain contained within the amino-terminal half of the heavy chain (H_{N}) that facilitates release of the LC from intracellular vesicles into the cytoplasm of the target cell; and 3) a binding domain found within the carboxyl-terminal half of the heavy chain (H_{C}) that determines the binding activity and binding specificity of the toxin to the receptor complex located at the surface of the target cell. The H_{C} domain comprises two distinct structural features of roughly equal size that indicate function and are designated the H_{CN} and H_{CC} subdomains.

Clostridial toxins act on the nervous system by blocking the release of acetylcholine (ACh) at the pre-synaptic neuromuscular junction. The binding, translocation and enzymatic activity of these three functional domains are all necessary for toxicity. While all details of this process are not yet precisely known, the overall cellular intoxication mechanism whereby Clostridial toxins enter a neuron and inhibit neurotransmitter release is similar, regardless of serotype or subtype. Although applicants have no wish to be limited by the following description, the intoxication mechanism can be described as comprising at least four steps: 1) receptor binding, 2) complex internalization, 3) light chain translocation, and 4) enzymatic target modification. The process is initiated when the binding domain of a Clostridial toxin binds to a toxin-specific receptor system located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the toxin/receptor complexes are internalized by endocytosis and the internalized vesicles are sorted to specific intracellular routes. The translocation step appears to be triggered by the acidification of the vesicle compartment. This process seems to initiate pH-dependent structural rearrangements that increase hydrophobicity, create a pore in the vesicle membrane, and promote formation of the di-chain form of the toxin. Once di-chain formation occurs, light chain endopeptidase of the toxin is released from the intracellular vesicle via the pore into the cytosol where it appears to specifically target one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface.

Aspects of the present specification disclose, in part, a Clostridial toxin. As used herein, the term “Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/C₁, a BoNT/D, a BoNT/E, a BoNT/F, a BoNT/G, a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof.

A Clostridial toxin disclosed herein also includes a recombinantly-produced Clostridial toxins. Non-limiting examples of recombinantly-produced Clostridial toxins include a recombinantly-produced BoNT like BoNT/A, a BoNT/B, a BoNT/C₁, a BoNT/D, a BoNT/E, a BoNT/F, a BoNT/G, a recombinantly-produced TeNT, a recombinantly-produced BaNT, and a recombinantly-produced BuNT.

A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

Clostridial toxins can be produced using standard purification or recombinant biology techniques known to those skilled in the art. See, e.g., Hui Xiang et al., Animal Product Free System and Process for Purifying a Botulinum Toxin, U.S. Pat. No. 7,354,740, entirely incorporated by reference. For example, a BoNT/A complex can be isolated and purified from an anaerobic fermentation by cultivating Clostridium botulinum type A in a suitable medium. Raw toxin can be harvested by precipitation with sulfuric acid and concentrated by ultramicrofiltration. Purification can be carried out by dissolving the acid precipitate in calcium chloride. The toxin can then be precipitated with cold ethanol. The precipitate can be dissolved in sodium phosphate buffer and centrifuged. Upon drying there can then be obtained approximately 900 kD crystalline BoNT/A complex with a specific potency of 3×10⁷ LD₅₀ U/mg or greater. Furthermore, NAPs can be separated out to obtain purified toxin, such as e.g., BoNT/A with an approximately 150 kD molecular weight with a specific potency of 1−2×10⁸ LD₅₀ U/mg or greater, purified BoNT/B with an approximately 156 kD molecular weight with a specific potency of 1−2×10⁸ LD₅₀ U/mg or greater, and purified BoNT/F with an approximately 155 kD molecular weight with a specific potency of 1−2×10⁷ LD₅₀ U/mg or greater. See Edward J. Schantz & Eric A. Johnson, Properties and use of Botulinum Toxin and Other Microbial Neurotoxins in Medicine, Microbiol Rev. 56: 80-99 (1992), incorporated entirely by reference. As another example, recombinant Clostridial toxins can be recombinantly produced as described in Steward et al., Optimizing Expression of Active Botulinum Toxin Type A, U.S. Patent Publication 2008/0057575; and Steward et al., Optimizing Expression of Active Botulinum Toxin Type E, U.S. Patent Publication 2008/0138893, each incorporated entirety by reference.

Clostridial toxins are also commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea), BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc., South San Francisco, Calif.). Clostridial toxin complexes may be obtained from, e.g., List Biological Laboratories, Inc. (Campbell, Calif.), the Centre for Applied Microbiology and Research (Porton Down, U.K), Wako (Osaka, Japan), and Sigma Chemicals (St Louis, Mo.).

Aspects of the present specification disclose, in part, a Targeted Exocytosis Modulator. As used herein, the term “Targeted Exocytosis Modulator” is synonymous with “TEM”, “Targeted Vesicular Exocytosis Modulator Protein”, “TVEMP”, or “retargeted endopeptidase.” Generally, a TEM comprises an enzymatic domain from a Clostridial toxin light chain, a translocation domain from a Clostridial toxin heavy chain, and a targeting domain. The targeting domain of a TEM provides an altered cell targeting capability that targets the molecule to a receptor other than the native Clostridial toxin receptor utilized by a naturally-occurring Clostridial toxin. For example, a TEM can target a sensory neuron, a sympathetic neuron, and/or a parasympathetic neuron. This re-targeted capability is achieved by replacing the naturally-occurring binding domain of a Clostridial toxin with a targeting domain having a binding activity for a non-Clostridial toxin receptor. Although binding to a non-Clostridial toxin receptor, a TEM undergoes all the other steps of the intoxication process including internalization of the TEM/receptor complex into the cytoplasm, formation of the pore in the vesicle membrane and di-chain molecule, translocation of the enzymatic domain into the cytoplasm, and exerting a proteolytic effect on a component of the SNARE complex of the target cell.

An important difference between TEMs, such as TEMs disclosed herein, and native Clostridial toxins is that since TEMs do not target motor neurons, the lethality associated with over-dosing an individual with a TEM is greatly minimized, if not avoided altogether. For example, a TEM comprising an opioid targeting domain can be administered at 10,000 times the therapeutically effective dose before evidence of lethality is observed, and this lethality is due to the passive diffusion of the molecule and not via the intoxication process. Thus, for all practical purposes TEMs are non-lethal molecules.

As used herein, the term “Clostridial toxin enzymatic domain” refers to a Clostridial toxin polypeptide located in the light chain of a Clostridial toxin that executes the enzymatic target modification step of the intoxication process. A Clostridial toxin enzymatic domain includes a metalloprotease region containing a zinc-dependent endopeptidase activity which specifically targets core components of the neurotransmitter release apparatus. Thus, a Clostridial toxin enzymatic domain specifically targets and proteolytically cleavages of a Clostridial toxin substrate, such as, e.g., SNARE proteins like a SNAP-25 substrate, a VAMP substrate and a Syntaxin substrate.

A Clostridial toxin enzymatic domain includes, without limitation, naturally occurring Clostridial toxin enzymatic domain variants, such as, e.g., Clostridial toxin enzymatic domain isoforms and Clostridial toxin enzymatic domain subtypes; non-naturally occurring Clostridial toxin enzymatic domain variants, such as, e.g., conservative Clostridial toxin enzymatic domain variants, non-conservative Clostridial toxin enzymatic domain variants, Clostridial toxin enzymatic domain chimeras, active Clostridial toxin enzymatic domain fragments thereof, or any combination thereof. Non-limiting examples of a Clostridial toxin enzymatic domain include, e.g., a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, and a BuNT enzymatic domain.

As used herein, the term “Clostridial toxin translocation domain” refers to a Clostridial toxin polypeptide located within the amino-terminal half of the heavy chain of a Clostridial toxin that executes the translocation step of the intoxication process. The translocation step appears to involve an allosteric conformational change of the translocation domain caused by a decrease in pH within the intracellular vesicle. This conformational change results in the formation of a pore in the vesicular membrane that permits the movement of the light chain from within the vesicle into the cytoplasm. Thus, a Clostridial toxin translocation domain facilitates the movement of a Clostridial toxin light chain across a membrane of an intracellular vesicle into the cytoplasm of a cell.

A Clostridial toxin translocation domain includes, without limitation, naturally occurring Clostridial toxin translocation domain variants, such as, e.g., Clostridial toxin translocation domain isoforms and Clostridial toxin translocation domain subtypes; non-naturally occurring Clostridial toxin translocation domain variants, such as, e.g., conservative Clostridial toxin translocation domain variants, non-conservative Clostridial toxin translocation domain variants, Clostridial toxin translocation domain chimerics, active Clostridial toxin translocation domain fragments thereof, or any combination thereof. Non-limiting examples of a Clostridial toxin translocation domain include, e.g., a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, and a BuNT translocation domain.

As used herein, the term “targeting domain” is synonymous with “binding domain” or “targeting moiety” and refers to a polypeptide that executes the receptor binding and/or complex internalization steps of the intoxication process, with the proviso that the binding domain is not a Clostridial toxin binding domain found within the carboxyl-terminal half of the heavy chain of a Clostridial toxin. A targeting domain includes a receptor binding region that confers the binding activity and/or specificity of the targeting domain for its cognate receptor. As used herein, the term “cognate receptor” refers to a receptor for which the targeting domain preferentially interacts with under physiological conditions, or under in vitro conditions substantially approximating physiological conditions. As used herein, the term “preferentially interacts” is synonymous with “preferentially binding” and refers to an interaction that is statistically significantly greater in degree relative to a control. With reference to a targeting domain disclosed herein, a targeting domain binds to its cognate receptor to a statistically significantly greater degree relative to a non-cognate receptor. Said another way, there is a discriminatory binding of the targeting domain to its cognate receptor relative to a non-cognate receptor. Thus, a targeting domain directs binding to a TEM-specific receptor located on the plasma membrane surface of a target cell.

In an embodiment, a targeting domain disclosed herein has an association rate constant that confers preferential binding to its cognate receptor. In aspects of this embodiment, a targeting domain disclosed herein binds to its cognate receptor with an association rate constant of, e.g., less than 1×10⁵ M⁻¹ s⁻¹, less than 1×10⁶ M⁻¹ s⁻¹, less than 1×10⁷ M⁻¹ s⁻¹, or less than 1×10⁸ M⁻¹ s⁻¹. In other aspects of this embodiment, a targeting domain disclosed herein binds to its cognate receptor with an association rate constant of, e.g., more than 1×10⁵ M⁻¹ s⁻¹, more than 1×10⁶ M⁻¹ s⁻¹, more than 1×10⁷ M⁻¹ s⁻¹, or more than 1×10⁸ M⁻¹ s⁻¹. In yet other aspects of this embodiment, a targeting domain disclosed herein binds to its cognate receptor with an association rate constant between 1×10⁵ M⁻¹ s⁻¹ to 1×10⁸ s⁻¹, 1×10⁶ M⁻¹ s⁻¹ to 1×10⁸ M⁻¹ s⁻¹, 1×10⁵ M⁻¹ s⁻¹ to 1×10⁷ M⁻¹ s⁻¹, or 1×10⁶ M⁻¹ s⁻¹ to 1×10⁷ M⁻¹ s⁻¹.

In another embodiment, a targeting domain disclosed herein has an association rate constant that is greater for its cognate target receptor relative to a non-cognate receptor. In other aspects of this embodiment, a targeting domain disclosed herein has an association rate constant that is greater for its cognate target receptor relative to a non-cognate receptor by, at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has an association rate constant that is greater for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10.000-fold, or about 10-fold to about 100.000-fold.

In yet another embodiment, a targeting domain disclosed herein has a disassociation rate constant that confers preferential binding to its cognate receptor. In other aspects of this embodiment, a targeting domain disclosed herein binds to its cognate receptor with a disassociation rate constant of less than 1×10⁻³ s⁻¹, less than 1×10⁻⁴ s⁻¹, or less than 1×10⁻⁵ s⁻¹. In yet other aspects of this embodiment, a targeting domain disclosed herein binds to its cognate receptor with a disassociation rate constant of, e.g., less than 1.0×10⁻⁴ s⁻¹, less than 2.0×10⁻⁴ s⁻¹, less than 3.0×10⁻⁴ s⁻¹, less than 4.0×10⁻⁴ s⁻¹, less than 5.0×10⁻⁴ s⁻¹, less than 6.0×10⁻⁴ s⁻¹, less than 7.0×10⁻⁴ s⁻¹, less than 8.0×10⁻⁴ s⁻¹, or less than 9.0×10⁻⁴ s⁻¹. In still other aspects of this embodiment, a targeting domain disclosed herein binds to its cognate receptor with a disassociation rate constant of, e.g., more than 1×10⁻³ s⁻¹, more than 1×10⁻⁴ s⁻¹, or more than 1×10⁻⁵ s⁻¹. In other aspects of this embodiment, a targeting domain disclosed herein binds to its cognate receptor with a disassociation rate constant of, e.g., more than 1.0×10⁻⁴ s⁻¹, more than 2.0×10⁻⁴ s⁻¹, more than 3.0×10⁻⁴ s⁻¹, more than 4.0×10⁻⁴ s⁻¹, more than 5.0×10⁻⁴ s⁻¹, more than 6.0×10⁻⁴ s⁻¹, more than 7.0×10⁻⁴ s⁻¹, more than 8.0×10⁻⁴ s⁻¹, or more than 9.0×10⁻⁴ s⁻¹.

In still another embodiment, a targeting domain disclosed herein has a disassociation rate constant that is less for its cognate target receptor relative to a non-cognate receptor. In other aspects of this embodiment, a targeting domain disclosed herein has a disassociation rate constant that is less for its cognate target receptor relative to a non-cognate receptor by, e.g., at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has a disassociation rate constant that is less for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10.000-fold, or about 10-fold to about 100.000-fold.

In another embodiment, a targeting domain disclosed herein has an equilibrium disassociation constant that confers preferential binding to its cognate receptor. In other aspects of this embodiment, a targeting domain disclosed herein binds to its cognate receptor with an equilibrium disassociation constant of, e.g., less than 0.500 nM. In yet other aspects of this embodiment, a targeting domain disclosed herein binds to its cognate receptor with an equilibrium disassociation constant of, e.g., less than 0.500 nM, less than 0.450 nM, less than 0.400 nM, less than 0.350 nM, less than 0.300 nM, less than 0.250 nM, less than 0.200 nM, less than 0.150 nM, less than 0.100 nM, or less than 0.050 nM. In other aspects of this embodiment, a targeting domain disclosed herein binds to its cognate receptor with an equilibrium disassociation constant of, e.g., more than 0.500 nM, more than 0.450 nM, more than 0.400 nM, more than 0.350 nM, more than 0.300 nM, more than 0.250 nM, more than 0.200 nM, more than 0.150 nM, more than 0.100 nM, or more than 0.050 nM.

In yet another embodiment, a targeting domain disclosed herein has an equilibrium disassociation constant that is greater for its cognate target receptor relative to a non-cognate receptor. In other aspects of this embodiment, a targeting domain disclosed herein has an equilibrium disassociation constant that is greater for its cognate target receptor relative to a non-cognate receptor by, e.g., at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has an equilibrium disassociation constant that is greater for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10.000-fold, or about 10-fold to about 100.000-fold.

In another embodiment, a targeting domain disclosed herein may be one that preferentially interacts with a receptor located on a sensory neuron. In an aspect of this embodiment, the sensory neuron targeting domain is one whose cognate receptor is located exclusively on the plasma membrane of sensory neurons. In another aspect of this embodiment, the sensory neuron targeting domain is one whose cognate receptor is located primarily on the plasma membrane of sensory neuron. For example, a receptor for a sensory neuron targeting domain is located primarily on a sensory neuron when, e.g., at least 60% of all cells that have a cognate receptor for a sensory neuron targeting domain on the surface of the plasma membrane are sensory neurons, at least 70% of all cells that have a cognate receptor for a sensory neuron targeting domain on the surface of the plasma membrane are sensory neurons, at least 80% of all cells that have a cognate receptor for a sensory neuron targeting domain on the surface of the plasma membrane are sensory neurons, or at least 90% of all cells that have a cognate receptor for a sensory neuron targeting domain on the surface of the plasma membrane are sensory neurons. In yet another aspect of this embodiment, the sensory neuron targeting domain is one whose cognate receptor is located on the plasma membrane of several types of cells, including sensory neurons. In still another aspect of this embodiment, the sensory neuron targeting domain is one whose cognate receptor is located on the plasma membrane of several types of cells, including sensory neurons, with the proviso that motor neurons are not one of the other types of cells.

In another embodiment, a targeting domain disclosed herein may be one that preferentially interacts with a receptor located on a sympathetic neuron. In an aspect of this embodiment, the sympathetic neuron targeting domain is one whose cognate receptor is located exclusively on the plasma membrane of sympathetic neurons. In another aspect of this embodiment, the sympathetic neuron targeting domain is one whose cognate receptor is located primarily on the plasma membrane of sympathetic neuron. For example, a receptor for a sympathetic neuron targeting domain is located primarily on a sympathetic neuron when, e.g., at least 60% of all cells that have a cognate receptor for a sympathetic neuron targeting domain on the surface of the plasma membrane are sympathetic neurons, at least 70% of all cells that have a cognate receptor for a sympathetic neuron targeting domain on the surface of the plasma membrane are sympathetic neurons, at least 80% of all cells that have a cognate receptor for a sympathetic neuron targeting domain on the surface of the plasma membrane are sympathetic neurons, or at least 90% of all cells that have a cognate receptor for a sympathetic neuron targeting domain on the surface of the plasma membrane are sympathetic neurons. In yet another aspect of this embodiment, the sympathetic neuron targeting domain is one whose cognate receptor is located on the plasma membrane of several types of cells, including sympathetic neurons. In still another aspect of this embodiment, the sympathetic neuron targeting domain is one whose cognate receptor is located on the plasma membrane of several types of cells, including sympathetic neurons, with the proviso that motor neurons are not one of the other types of cells.

In another embodiment, a targeting domain disclosed herein may be one that preferentially interacts with a receptor located on a parasympathetic neuron. In an aspect of this embodiment, the parasympathetic neuron targeting domain is one whose cognate receptor is located exclusively on the plasma membrane of parasympathetic neurons. In another aspect of this embodiment, the parasympathetic neuron targeting domain is one whose cognate receptor is located primarily on the plasma membrane of parasympathetic neuron. For example, a receptor for a parasympathetic neuron targeting domain is located primarily on a parasympathetic neuron when, e.g., at least 60% of all cells that have a cognate receptor for a parasympathetic neuron targeting domain on the surface of the plasma membrane are parasympathetic neurons, at least 70% of all cells that have a cognate receptor for a parasympathetic neuron targeting domain on the surface of the plasma membrane are parasympathetic neurons, at least 80% of all cells that have a cognate receptor for a parasympathetic neuron targeting domain on the surface of the plasma membrane are parasympathetic neurons, or at least 90% of all cells that have a cognate receptor for a parasympathetic neuron targeting domain on the surface of the plasma membrane are parasympathetic neurons. In yet another aspect of this embodiment, the parasympathetic neuron targeting domain is one whose cognate receptor is located on the plasma membrane of several types of cells, including parasympathetic neurons. In still another aspect of this embodiment, the parasympathetic neuron targeting domain is one whose cognate receptor is located on the plasma membrane of several types of cells, including parasympathetic neurons, with the proviso that motor neurons are not one of the other types of cells.

In another embodiment, a targeting domain disclosed herein is an opioid peptide targeting domain, a galanin peptide targeting domain, a PAR peptide targeting domain, a somatostatin peptide targeting domain, a neurotensin peptide targeting domain, a SLURP peptide targeting domain, an angiotensin peptide targeting domain, a tachykinin peptide targeting domain, a Neuropeptide Y related peptide targeting domain, a kinin peptide targeting domain, a melanocortin peptide targeting domain, or a granin peptide targeting domain, a glucagon like hormone peptide targeting domain, a secretin peptide targeting domain, a pituitary adenylate cyclase activating peptide (PACAP) peptide targeting domain, a growth hormone-releasing hormone (GHRH) peptide targeting domain, a vasoactive intestinal peptide (VIP) peptide targeting domain, a gastric inhibitory peptide (GIP) peptide targeting domain, a calcitonin peptide targeting domain, a visceral gut peptide targeting domain, a neurotrophin peptide targeting domain, a head activator (HA) peptide, a glial cell line-derived neurotrophic factor (GDNF) family of ligands (GFL) peptide targeting domain, a RF-amide related peptide (RFRP) peptide targeting domain, a neurohormone peptide targeting domain, or a neuroregulatory cytokine peptide targeting domain, an interleukin (IL) targeting domain, vascular endothelial growth factor (VEGF) targeting domain, an insulin-like growth factor (IGF) targeting domain, an epidermal growth factor (EGF) targeting domain, a Transformation Growth Factor-β (TGFβ) targeting domain, a Bone Morphogenetic Protein (BMP) targeting domain, a Growth and Differentiation Factor (GDF) targeting domain, an activin targeting domain, or a Fibroblast Growth Factor (FGF) targeting domain, or a Platelet-Derived Growth Factor (PDGF) targeting domain.

In an aspect of this embodiment, an opioid peptide targeting domain is an enkephalin peptide, a bovine adrenomedullary-22 (BAM22) peptide, an endomorphin peptide, an endorphin peptide, a dynorphin peptide, a nociceptin peptide, or a hemorphin peptide. In another aspect of this embodiment, an enkephalin peptide targeting domain is a Leu-enkephalin peptide, a Met-enkephalin peptide, a Met-enkephalin MRGL peptide, or a Met-enkephalin MRF peptide. In another aspect of this embodiment, a bovine adrenomedullary-22 peptide targeting domain is a BAM22 (1-12) peptide, a BAM22 (6-22) peptide, a BAM22 (8-22) peptide, or a BAM22 (1-22) peptide. In another aspect of this embodiment, an endomorphin peptide targeting domain is an endomorphin-1 peptide or an endomorphin-2 peptide. In another aspect of this embodiment, an endorphin peptide targeting domain an endorphin-α peptide, a neoendorphin-α peptide, an endorphin-β peptide, a neoendorphin-β peptide, or an endorphin-γ peptide. In another aspect of this embodiment, a dynorphin peptide targeting domain is a dynorphin A peptide, a dynorphin B (leumorphin) peptide, or a rimorphin peptide. In another aspect of this embodiment, a nociceptin peptide targeting domain is a nociceptin RK peptide, a nociceptin peptide, a neuropeptide 1 peptide, a neuropeptide 2 peptide, or a neuropeptide 3 peptide. In another aspect of this embodiment, a hemorphin peptide targeting domain is a LVVH7 peptide, a VVH7 peptide, a VH7 peptide, a H7 peptide, a LVVH6 peptide, a LVVH5 peptide, a VVH5 peptide, a LVVH4 peptide, or a LVVH3 peptide.

In an aspect of this embodiment, a galanin peptide targeting domain is a galanin peptide, a galanin message-associated peptide (GMAP) peptide, a galanin like protein (GALP) peptide, or an alarin peptide.

In an aspect of this embodiment, a PAR peptide targeting domain is a PAR1 peptide, a PAR2 peptide, a PAR3 peptide and a PAR4 peptide. In an aspect of this embodiment, a somatostatin peptide targeting domain is a somatostatin peptide or a cortistatin peptide. In an aspect of this embodiment, a neurotensin peptide targeting domain a neurotensin or a neuromedin N. In an aspect of this embodiment, a SLURP peptide targeting domain is a SLURP-1 peptide or a SLURP-2 peptide. In an aspect of this embodiment, an angiotensin peptide targeting domain is an angiotensin peptide.

In an aspect of this embodiment, a tachykinin peptide targeting domain is a Substance P peptide, a neuropeptide K peptide, a neuropeptide gamma peptide, a neurokinin A peptide, a neurokinin B peptide, a hemokinin peptide, or a endokinin peptide. In an aspect of this embodiment, a Neuropeptide Y related peptide targeting domain is a Neuropeptide Y peptide, a Peptide YY peptide, Pancreatic peptide peptide, a Pancreatic icosapeptide peptide, a Pancreatic Hormone domain peptide, a CXCL12 peptide, and a Sjogren syndrome antigen B peptide. In an aspect of this embodiment, a kinin peptide targeting domain is a bradykinin peptide, a kallidin peptide, a desArg9 bradykinin peptide, a desArg10 bradykinin peptide, a kininogen peptide, gonadotropin releasing hormone 1 peptide, chemokine peptide, an arginine vasopressin peptide.

In an aspect of this embodiment, a melanocortin peptide targeting domain comprises a melanocyte stimulating hormone peptide, an adrenocorticotropin peptide, a lipotropin peptide, or a melanocortin peptide derived neuropeptide. In an aspect of this embodiment, a melanocyte stimulating hormone peptide targeting domain comprises an α-melanocyte stimulating hormone peptide, a β-melanocyte stimulating hormone peptide, or a γ-melanocyte stimulating hormone peptide. In an aspect of this embodiment, an adrenocorticotropin peptide targeting domain comprises an adrenocorticotropin or a Corticotropin-like intermediary peptide. In an aspect of this embodiment, a lipotropin peptide targeting domain comprises a β-lipotropin peptide or a γ-lipotropin peptide.

In an aspect of this embodiment, a granin peptide targeting domain comprises a chromogranin A peptide, a chromogranin B peptide, a chromogranin C (secretogranin II) peptide, a secretogranin IV peptide, or a secretogranin VI peptide. In an aspect of this embodiment, a chromogranin A peptide targeting domain comprises a β-granin peptide, a vasostatin peptide, a chromostatin peptide, a pancreastatin peptide, a WE-14 peptide, a catestatin peptide, a parastatin peptide, or a GE-25 peptide. In an aspect of this embodiment, a chromogranin B peptide targeting domain comprises a GAWK peptide, an adrenomedullary peptide, or a secretolytin peptide. In an aspect of this embodiment, a chromogranin C peptide targeting domain comprises a secretoneurin peptide.

In an aspect of this embodiment, a glucagons-like hormone peptide targeting domain is a glucagon-like peptide-1, a glucagon-like peptide-2, a glicentin, a glicentin-related peptide (GRPP), a glucagon, or an oxyntomodulin (OXY). In an aspect of this embodiment, a secretin peptide targeting domain is a secretin peptide. In an aspect of this embodiment, a pituitary adenylate cyclase activating peptide targeting domain is a pituitary adenylate cyclase activating peptide. In an aspect of this embodiment, a growth hormone-releasing hormone peptide targeting domain a growth hormone-releasing hormone peptide. In an aspect of this embodiment, a vasoactive intestinal peptide targeting domain is a vasoactive intestinal peptide-1 peptide or a vasoactive intestinal peptide-2 peptide. In an aspect of this embodiment, a gastric inhibitory peptide targeting domain is a gastric inhibitory peptide. In an aspect of this embodiment, a calcitonin peptide targeting domain is a calcitonin peptide, an amylin peptide, a calcitonin-related peptide α, a calcitonin-related peptide β, and a islet amyloid peptide. In an aspect of this embodiment, a visceral gut peptide targeting domain is a gastrin peptide, a gastrin-releasing peptide, or a cholecystokinin peptide.

In an aspect of this embodiment, a neurotrophin peptide targeting domain is a nerve growth factor (NGF) peptide, a brain derived neurotrophic factor (BDNF) peptide, a neurotrophin-3 (NT-3) peptide, a neurotrophin-4/5 (NT-4/5) peptide, or an amyloid beta (A4) precursor protein neurotrophin (APP) peptide. In an aspect of this embodiment, a head activator peptide targeting domain is a head activator peptide. In an aspect of this embodiment, a glial cell line-derived neurotrophic factor family of ligands peptide targeting domain is a glial cell line-derived neurotrophic factor peptide, a Neurturin peptide, a Persephrin peptide, or an Artemin peptide. In an aspect of this embodiment, a RF-amide related peptide targeting domain a RF-amide related peptide-1, a RF-amide related peptide-2, a RF-amide related peptide-3, a neuropeptide AF, or a neuropeptide FF.

In an aspect of this embodiment, a neurohormone peptide targeting domain is a corticotropin-releasing hormone (CCRH), a parathyroid hormone (PTH), a parathyroid hormone-like hormone (PTHLH), a PHYH, a thyrotropin-releasing hormone (TRH), an urocortin-1 (UCN1), an urocortin-2 (UCN2), an urocortin-3 (UCN3), or an urotensin 2 (UTS2). In an aspect of this embodiment, a neuroregulatory cytokine peptide targeting domain is a ciliary neurotrophic factor peptide, a glycophorin-A peptide, a leukemia inhibitory factor peptide, a cardiotrophin-1 peptide, a cardiotrophin-like cytokine peptide, a neuroleukin peptide, and an onostatin M peptide. In an aspect of this embodiment, an IL peptide targeting domain is an IL-1 peptide, an IL-2 peptide, an IL-3 peptide, an IL-4 peptide, an IL-5 peptide, an IL-6 peptide, an IL-7 peptide, an IL-8 peptide, an IL-9 peptide, an IL-10 peptide, an IL-11 peptide, an IL-12 peptide, an IL-18 peptide, an IL-32 peptide, or an IL-33 peptide.

In an aspect of this embodiment, a VEGF peptide targeting domain is a VEGF-A peptide, a VEGF-B peptide, a VEGF-C peptide, a VEGF-D peptide, or a placenta growth factor (PIGF) peptide. In an aspect of this embodiment, an IGF peptide targeting domain is an IGF-1 peptide or an IGF-2 peptide. In an aspect of this embodiment, an EGF peptide targeting domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4). In an aspect of this embodiment, a FGF peptide targeting domain is a FGF1 peptide, a FGF2 peptide, a FGF3 peptide, a FGF4 peptide, a FGF5 peptide, a FGF6 peptide, a FGF7 peptide, a FGF8 peptide, a FGF9 peptide, a FGF10 peptide, a FGF17 peptide, or a FGF18 peptide. In an aspect of this embodiment, a PDGF peptide targeting domain is a PDGFα peptide or a PDGβ3 peptide.

In an aspect of this embodiment, a TGFβ peptide targeting domain is a TGFβ1 peptide, a TGFβ2 peptide, a TGFβ3 peptide, or a TGFβ4 peptide. In an aspect of this embodiment, a BMP peptide targeting domain is a BMP2 peptide, a BMP3 peptide, a BMP4 peptide, a BMP5 peptide, a BMP6 peptide, a BMP7 peptide, a BMP8 peptide, or a BMP10 peptide. In an aspect of this embodiment, a GDF peptide targeting domain is a GDF1 peptide, a GDF2 peptide, a GDF3 peptide, a GDF5 peptide, a GDF6 peptide, a GDF7 peptide, a GDF8 peptide, a GDF10 peptide, a GDF11 peptide, or a GDF15 peptide. In an aspect of this embodiment, an activin peptide targeting domain is an activin A peptide, an activin B peptide, an activin C peptide, an activin E peptide, or an inhibin A peptide.

As discussed above, naturally-occurring Clostridial toxins are organized into three functional domains comprising a linear amino-to-carboxyl single polypeptide order of the enzymatic domain (amino region position), the translocation domain (middle region position) and the binding domain (carboxyl region position). This naturally-occurring order can be referred to as the carboxyl presentation of the binding domain because the domain necessary for binding to the receptor is located at the carboxyl region position of the Clostridial toxin. However, it has been shown that Clostridial toxins can be modified by rearranging the linear amino-to-carboxyl single polypeptide order of the three major domains and locating a targeting moiety at the amino region position of a Clostridial toxin, referred to as amino presentation, as well as in the middle region position, referred to as central presentation.

Thus, a TEM can comprise a targeting domain in any and all locations with the proviso that TEM is capable of performing the intoxication process. Non-limiting examples include, locating a targeting domain at the amino terminus of a TEM; locating a targeting domain between a Clostridial toxin enzymatic domain and a Clostridial toxin translocation domain of a TEM; and locating a targeting domain at the carboxyl terminus of a TEM. Other non-limiting examples include, locating a targeting domain between a Clostridial toxin enzymatic domain and a Clostridial toxin translocation domain of a TEM. The enzymatic domain of naturally-occurring Clostridial toxins contains the native start methionine. Thus, in domain organizations where the enzymatic domain is not in the amino-terminal location an amino acid sequence comprising the start methionine should be placed in front of the amino-terminal domain. Likewise, where a targeting domain is in the amino-terminal position, an amino acid sequence comprising a start methionine and a protease cleavage site may be operably-linked in situations in which a targeting domain requires a free amino terminus, see, e.g., Shengwen Li et al., Degradable Clostridial Toxins, U.S. patent application Ser. No. 11/572,512, entirely incorporated by reference. In addition, it is known in the art that when adding a polypeptide that is operably-linked to the amino terminus of another polypeptide comprising the start methionine that the original methionine residue can be deleted.

A TEM disclosed herein may optionally comprise an exogenous protease cleavage site that allows the use of an exogenous protease to convert the single-chain polypeptide form of a TEM into its more active di-chain form. As used herein, the term “exogenous protease cleavage site” is synonymous with a “non-naturally occurring protease cleavage site” or “non-native protease cleavage site” and means a protease cleavage site that is not naturally found in a di-chain loop region from a naturally occurring Clostridial toxin.

Naturally-occurring Clostridial toxins are each translated as a single-chain polypeptide of approximately 150 kDa that is subsequently cleaved by proteolytic scission within a disulfide loop by a naturally-occurring protease. This cleavage occurs within the discrete di-chain loop region located between two cysteine residues that form a disulfide bridge and comprising an endogenous protease cleavage site. As used herein, the term “endogenous di-chain loop protease cleavage site” is synonymous with a “naturally occurring di-chain loop protease cleavage site” and refers to a naturally occurring protease cleavage site found within the di-chain loop region of a naturally occurring Clostridial toxin. This posttranslational processing yields a di-chain molecule comprising an approximately 50 kDa light chain, comprising the enzymatic domain, and an approximately 100 kDa heavy chain, comprising the translocation and cell binding domains, the light chain and heavy chain being held together by the single disulfide bond and non-covalent interactions. Recombinantly-produced Clostridial toxins generally substitute the naturally-occurring di-chain loop protease cleavage site with an exogenous protease cleavage site to facilitate production of a recombinant di-chain molecule. See e.g., Dolly, J. O. et al., Activatable Clostridial Toxins, U.S. Pat. No. 7,419,676, incorporated entirely by reference.

Although TEMs vary in their overall molecular weight because the size of the targeting domain, the activation process and its reliance on an exogenous cleavage site is essentially the same as that for recombinantly-produced Clostridial toxins. See e.g., Steward, et al., Activatable Clostridial Toxins, US 2009/0081730; Steward, et al., Modified Clostridial Toxins with Enhanced Translocation Capabilities and Altered Targeting Activity For Non-Clostridial Toxin Target Cells, U.S. patent application Ser. No. 11/776,075; Steward, et al., Modified Clostridial Toxins with Enhanced Translocation Capabilities and Altered Targeting Activity for Clostridial Toxin Target Cells, US 2008/0241881, each incorporated entirely by reference. In general, the activation process that converts the single-chain polypeptide into its di-chain form using exogenous proteases can be used to process TEMs having a targeting domain organized in an amino presentation, central presentation, or carboxyl presentation arrangement. This is because for most targeting domains the amino-terminus of the moiety does not participate in receptor binding. As such, a wide range of protease cleavage sites can be used to produce an active di-chain form of a TEM. However, targeting domains requiring a free amino-terminus for receptor binding require a protease cleavage site whose scissile bond is located at the carboxyl terminus. The use of protease cleavage site is the design of a TEM are described in, e.g., Steward, et al., Activatable Clostridial toxins, US 2009/0069238; Ghanshani, et al., Modified Clostridial Toxins Comprising an Integrated Protease Cleavage Site-Binding Domain, US 2011/0189162; and Ghanshani, et al., Methods of Intracellular Conversion of Single-Chain Proteins into their Di-chain Form, International Patent Application Serial No. PCT/US2011/22272, each of which is incorporated by reference in its entirety.

Non-limiting examples of exogenous protease cleavage sites include, e.g., a papain cleavage site like a plant papain cleavage site, an insect papain cleavage site, or a crustacian papain cleavage site, an enterokinase protease cleavage site, a Tobacco Etch Virus protease cleavage site, a Tobacco Vein Mottling Virus protease cleavage site, a human rhinovirus 3C protease cleavage site, a human enterovirus 3C protease cleavage site, a subtilisin cleavage site, a hydroxylamine cleavage site, a SUMO/ULP-1 protease cleavage site, and a Caspase 3 cleavage site.

Thus, in an embodiment, a TEM can comprise an amino to carboxyl single polypeptide linear order comprising a targeting domain, a translocation domain, an exogenous protease cleavage site and an enzymatic domain. In an aspect of this embodiment, a TEM can comprise an amino to carboxyl single polypeptide linear order comprising a targeting domain, a Clostridial toxin translocation domain, an exogenous protease cleavage site and a Clostridial toxin enzymatic domain.

In another embodiment, a TEM can comprise an amino to carboxyl single polypeptide linear order comprising a targeting domain, an enzymatic domain, an exogenous protease cleavage site, and a translocation domain. In an aspect of this embodiment, a TEM can comprise an amino to carboxyl single polypeptide linear order comprising a targeting domain, a Clostridial toxin enzymatic domain, an exogenous protease cleavage site, a Clostridial toxin translocation domain.

In yet another embodiment, a TEM can comprise an amino to carboxyl single polypeptide linear order comprising an enzymatic domain, an exogenous protease cleavage site, a targeting domain, and a translocation domain. In an aspect of this embodiment, a TEM can comprise an amino to carboxyl single polypeptide linear order comprising a Clostridial toxin enzymatic domain, an exogenous protease cleavage site, a targeting domain, and a Clostridial toxin translocation domain.

In yet another embodiment, a TEM can comprise an amino to carboxyl single polypeptide linear order comprising a translocation domain, an exogenous protease cleavage site, a targeting domain, and an enzymatic domain. In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

In another embodiment, a TEM can comprise an amino to carboxyl single polypeptide linear order comprising an enzymatic domain, a targeting domain, an exogenous protease cleavage site, and a translocation domain. In an aspect of this embodiment, a TEM can comprise an amino to carboxyl single polypeptide linear order comprising a Clostridial toxin enzymatic domain, a targeting domain, an exogenous protease cleavage site, a Clostridial toxin translocation domain.

In yet another embodiment, a TEM can comprise an amino to carboxyl single polypeptide linear order comprising a translocation domain, a targeting domain, an exogenous protease cleavage site and an enzymatic domain. In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

In still another embodiment, a TEM can comprise an amino to carboxyl single polypeptide linear order comprising an enzymatic domain, an exogenous protease cleavage site, a translocation domain, and a targeting domain. In an aspect of this embodiment, a TEM can comprise an amino to carboxyl single polypeptide linear order comprising a Clostridial toxin enzymatic domain, an exogenous protease cleavage site, a Clostridial toxin translocation domain, and a targeting domain.

In still another embodiment, a TEM can comprise an amino to carboxyl single polypeptide linear order comprising a translocation domain, an exogenous protease cleavage site, an enzymatic domain and a targeting domain. In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

Non-limiting examples of TEMs disclosed herein, including TEMs comprising a Clostridal toxin enzymatic domain, a Clostridial toxin translocation domain and a targeting domain, the use of an exogenous protease cleavage site, and the design of amino presentation, central presentation and carboxyl presentation TEMs are described in, e.g., U.S. Pat. No. 7,959,933, Activatable Recombinant Neurotoxins, U.S. Pat. No. 7,897,157, Activatable Clostridial Toxins; U.S. Pat. No. 7,833,535, Clostridial Toxin Derivatives and Methods for Treating Pain; U.S. Pat. No. 7,811,584, Multivalent Clostridial Toxins; U.S. Pat. No. 7,780,968, Clostridial Toxin Derivatives and Methods for Treating Pain; U.S. Pat. No. 7,749,514, Activatable Clostridial Toxins, U.S. Pat. No. 7,740,868, Activatable Clostridial Toxins; U.S. Pat. No. 7,736,659, Clostridial Toxin Derivatives and Methods for Treating Pain; U.S. Pat. No. 7,709,228, Activatable Recombinant Neurotoxins; U.S. Pat. No. 7,704,512, Clostridial Toxin Derivatives and Methods for Treating Pain; U.S. Pat. No. 7,659,092, Fusion Proteins; U.S. Pat. No. 7,658,933, Non-Cytotoxic Protein Conjugates; U.S. Pat. No. 7,622,127, Clostridial Toxin Derivatives and Methods for Treating Pain; U.S. Pat. No. 7,514,088, Multivalent Clostridial Toxin Derivatives and Methods of Their Use; U.S. Pat. No. 7,425,338, Clostridial Toxin Derivatives and Methods for Treating Pain; U.S. Pat. No. 7,422,877, Activatable Recombinant Neurotoxins; U.S. Pat. No. 7,419,676, Activatable Recombinant Neurotoxins; U.S. Pat. No. 7,413,742, Clostridial Toxin Derivatives and Methods for Treating Pain; U.S. Pat. No. 7,262,291, Clostridial Toxin Derivatives and Methods for Treating Pain; U.S. Pat. No. 7,244,437, Clostridial Toxin Derivatives and Methods for Treating Pain; U.S. Pat. No. 7,244,436, Clostridial Toxin Derivatives and Methods for Treating Pain; U.S. Pat. No. 7,138,127, Clostridial Toxin Derivatives and Methods for Treating Pain; U.S. Pat. No. 7,132,259, Activatable Recombinant Neurotoxins; U.S. Pat. No. 7,056,729, Botulinum Neurotoxin-Substance P Conjugate or Fusion Protein for Treating Pain; U.S. Pat. No. 6,641,820, Clostridial Toxin Derivatives and Methods to Treat Pain; U.S. Pat. No. 6,500,436, Clostridial Toxin Derivatives and Methods for Treating Pain; US 2011/0091437, Fusion Proteins; US 2011/0070621, Multivalent Clostridial Toxins; US 2011/0027256, Fusion Proteins; US 2010/0247509, Fusion Proteins; US 2010/0041098, Modified Clostridial Toxins with Altered Targeting Capabilities for Clostridial Toxin Target Cells; US 2010/0034802, Treatment of Pain; US 2009/0162341, Non-Cytotoxic Protein Conjugates; US 2009/0087458, Activatable Recombinant Neurotoxins; US 2009/0081730, Activatable Recombinant Neurotoxins; US 2009/0069238, Activatable Clostridial Toxins; US 2009/0042270, Activatable Recombinant Neurotoxins; US 2009/0030182, Activatable Recombinant Neurotoxins; US 2009/0018081, Activatable Clostridial Toxins; US 2009/0005313, Activatable Clostridial Toxins; US 2009/0004224, Activatable Clostridial Toxins; US 2008/0317783, Clostridial Toxin Derivatives and Methods for Treating Pain; US 2008/0241881, Modified Clostridial Toxins with Enhanced Translocation Capabilities and Altered Targeting Activity for Clostridial Toxin Target Cells; WO 2006/099590, Modified Clostridial Toxins with Altered Targeting Capabilities for Clostridial Toxin Target Cells; WO 2006/101809, Modified Clostridial Toxins with Enhanced Targeting Capabilities for Endogenous Clostridial Toxin Receptor Systems; WO 2007/106115, Modified Clostridial Toxins with Altered Targeting Capabilities for Clostridial Toxin Target Cells; WO 2008/008803, Modified Clostridial Toxins with Enhanced Translocation Capabilities and Altered Targeting Activity for Clostridial Toxin Target Cells; WO 2008/008805, Modified Clostridial Toxins with Enhanced Translocation Capabilities and Altered Targeting Activity For Non-Clostridial Toxin Target Cells; WO 2008/105901, Modified Clostridial Toxins with Enhanced Translocation Capability and Enhanced Targeting Activity; WO 2011/020052, Methods of Treating Cancer Using Opioid Retargeted Endpeptidases; WO 2011/020056, Methods of Treating Cancer Using Galanin Retargeted Endpeptidases; WO 2011/020114, Methods of Treating Cancer Using Tachykinin Retargeted Endopeptidases; WO 2011/020115, Methods of Treating Cancer Using Growth Factor Retargeted Endopeptidases; WO 2011/020117, Methods of Treating Cancer Using Neurotrophin Retargeted Endopeptidases; WO 2011/020119, Methods of Treating Cancer Using Glucagon-Like Hormone Retargeted Endopeptidases; each of which is incorporated by reference in its entirety.

Aspects of the present specification disclose, in part, a pharmaceutical composition comprising a Clostridial toxin and/or a TEM as disclosed herein. A composition disclosed herein is generally administered as a pharmaceutical acceptable composition. As used herein, the term “pharmaceutically acceptable” refers any molecular entity or composition that does not produce an adverse, allergic or other untoward or unwanted reaction when administered to an individual. As used herein, the term “pharmaceutically acceptable composition” is synonymous with “pharmaceutical composition” and means a therapeutically effective concentration of an active ingredient, such as, e.g., any of the Clostridial toxins and TEMs disclosed herein. A pharmaceutical composition disclosed herein is useful for medical and veterinary applications. A pharmaceutical composition may be administered to an individual alone, or in combination with other supplementary active ingredients, agents, drugs or hormones.

A Clostridial toxin and a TEM as disclosed herein may be provided as separate compositions or as part of a single composition. It is also understood that the two or more different Clostridial toxins and/or TEMs can be provided as separate compositions or as part of a single composition. Compositions disclosed herein can be useful in a method of treating bladder disorders disclosed herein.

A pharmaceutical composition comprising a Clostridial toxin and/or a TEM may optionally include a pharmaceutically acceptable carrier that facilitates processing of an active ingredient into pharmaceutically acceptable compositions. As used herein, the term “pharmacologically acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003).

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition. Exemplary pharmaceutical composition comprising a Clostridial toxin and a TEM are described in Hunt, et al., Animal Protein-Free Pharmaceutical Compositions, U.S. Ser. No. 12/331,816; and Dasari, et al., Clostridial Toxin Pharmaceutical Compositions, WO/2010/090677, each of entirely incorporated by reference.

In an embodiment, a composition is a pharmaceutical composition comprising a TEM. In aspects of this embodiment, a pharmaceutical composition comprising a TEM further comprises a pharmacological carrier, a pharmaceutical component, or both a pharmacological carrier and a pharmaceutical component. In other aspects of this embodiment, a pharmaceutical composition comprising a TEM further comprises at least one pharmacological carrier, at least one pharmaceutical component, or at least one pharmacological carrier and at least one pharmaceutical component.

In another embodiment, a composition is a pharmaceutical composition comprising a Clostridial toxin. In aspects of this embodiment, a pharmaceutical composition comprising a Clostridial toxin further comprises a pharmacological carrier, a pharmaceutical component, or both a pharmacological carrier and a pharmaceutical component. In other aspects of this embodiment, a pharmaceutical composition comprising a Clostridial toxin further comprises at least one pharmacological carrier, at least one pharmaceutical component, or at least one pharmacological carrier and at least one pharmaceutical component.

In yet another embodiment, a composition is a pharmaceutical composition comprising a Clostridial toxin and a TEM. In aspects of this embodiment, a pharmaceutical composition comprising a Clostridial toxin and a TEM further comprises a pharmacological carrier, a pharmaceutical component, or both a pharmacological carrier and a pharmaceutical component. In other aspects of this embodiment, a pharmaceutical composition comprising a Clostridial toxin and a TEM further comprises at least one pharmacological carrier, at least one pharmaceutical component, or at least one pharmacological carrier and at least one pharmaceutical component.

Aspects of the present specification disclose, in part, treating an individual suffering from a bladder disorder. As used herein, the term “treating,” refers to reducing or eliminating in an individual a clinical symptom of a bladder disorder; or delaying or preventing in an individual the onset of a clinical symptom of a bladder disorder. For example, the term “treating” can mean reducing a symptom of a condition characterized by a bladder disorder by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with a bladder disorder are well known and can be determined by a person of ordinary skill in the art by taking into account factors, including, without limitation, the location of the bladder disorder, the cause of the bladder disorder, the severity of the bladder disorder, and/or the tissue or organ affected by the bladder disorder. Treatment of a bladder disorder can also be indicated by a reduced need for a concurrent therapy. Those of skill in the art will know the appropriate symptoms or indicators associated with a specific type of bladder disorder and will know how to determine if an individual is a candidate for treatment as disclosed herein.

The pathophysioloogy of a bladder disorder can be neurogenic or idiopathic. A bladder disorder includes, without limitation, urinary incontinence, overactive bladder, detrusor dysfunction, lower urinary tract dysfunction, urinary retention, urinary hesitancy, polyuria, nocturia, chronic urinary tract infection, a bladder disorder associated with a prostate disorder, and a bladder disorder associated with a neurogenic dysfunction (such as, e.g., Parkinson's Disease, multiple sclerosis, spina bifida, transverse myelitis, stroke, spinal cord injury, spasm reflex, and a neurologic lesion of the spinal cord or brain).

An individual's ability to hold urine and maintain continence depends on normal function of the lower urinary tract, the kidneys, and the nervous system. The individual must also have a physical and psychological ability to recognize and appropriately respond to the urge to urinate. The bladders ability to fill and store urine requires a functional sphincter muscle (which controls the flow of urine out of the body) and a stable bladder wall muscle (detrusor). Normal bladder function is dependent on the nerves that sense the fullness of the bladder and on those that trigger the muscle movements that either empty it or retain urine. The process of urination involves two phases: 1) filling and storage of bladder and 2) emptying of bladder. During the filling and storage phase, the bladder stretches so it can hold the increasing amount of urine. The bladder of an average person can hold 350 mL to 550 mL of urine. Generally, the reflex to urinate is triggered when the bladder of an individual when approximately 200 mL of urine collects in the bladder. The emptying phase requires that the detrusor muscle contract, forcing urine out of the bladder through the urethra. The sphincter muscle must relax at the same time, so that urine can flow out of the body. The bladder, internal sphincters, and external sphincters may all be affected by aberrant motor and/or sensory nerve function that create abnormalities in bladder function. The aberrant neuronal activity can cause the bladder to be underactive, in which it is unable to contract and unable to empty completely, or it can be overactive, in which it contracts too quickly or frequently. Pain can also be associated with this improper function.

In one embodiment, a bladder disorder comprises a urinary incontinence. Urinary incontinence is the inability to control the passage of urine. This can range from an occasional leakage of urine, to a complete inability to hold any urine. Urinary incontinence can be caused by abnormalities in bladder capacity or malfunction of control mechanisms such as the bladder neck and/or external urethral sphincter muscle that are important for the bladders storage function. The many types of urinary incontinence.

In an aspect of this embodiment, an urinary incontinence is a stress incontinence. A stress incontinence is a type of urinary incontinence in which the strength of the muscles (urethral sphincter) that help control urination is reduced as a result of weakened pelvic muscles that support the bladder and urethra or because of malfunction of the urethral sphincter. The weakness may be caused by prior injury to the urethral area, neurological injury, some medications, or after surgery of the prostate or pelvic area. The sphincter is not able to prevent urine flow when there is increased pressure from the abdomen such as during certain activities like coughing, sneezing, laughing, or exercise. Stress urinary incontinence is the most common type of urinary incontinence in women. Studies have shown about 50% of all women have occasional urinary incontinence, and as many as 10% have frequent incontinence. Nearly 20% of women over age 75 experience daily urinary incontinence. Stress incontinence is often seen in women who have had multiple pregnancies and vaginal childbirths, whose bladder, urethra, or rectal wall stick out into the vaginal space (pelvic prolapse).

In another aspect of this embodiment, an urinary incontinence is an urge incontinence. An urge incontinence is a type of urinary incontinence that involves a strong, sudden need to urinate, followed by instant bladder contraction and involuntary loss of urine which results in leakage. There is not enough time between when an individual suffering from urge incontinence recognizes the need to urinate and when urination actually occurs. Urge incontinence is leakage of urine due to bladder muscles that contract inappropriately. Often these contractions occur regardless of the amount of urine that is in the bladder. Urge incontinence may result from neurological injuries (such as spinal cord injury or stroke), neurological dysfunction (such as, e.g., Parkinson's Disease and multiple sclerosis), infection, bladder cancer, bladder stones, bladder inflammation, or bladder outlet obstruction. In men, urge incontinence may be due to neurological disease or bladder changes caused by benign prostatic hypertrophy (BPH) or bladder outlet obstruction from an enlarged prostate. The majority of cases of urge incontinence are idiopathic, which means a specific cause cannot be identified. Although urge incontinence may occur in anyone at any age, it is more common in women and the elderly. Urge incontinence is also known as irritable bladder, spasmodic bladder, and unstable bladder.

In another aspect of this embodiment, an urinary incontinence is an overflow incontinence. An overflow urinary incontinence happens when small amounts of urine leak from a bladder that is always full. In older men, this can occur when the flow of urine from the bladder is blocked, usually by an enlarged prostate. It can sometimes be prevented by medication when early symptoms of prostate enlargement, such as frequent urination, appear. Some people with diabetes also have overflow incontinence. Mixed urinary incontinence describes a disorder where an individual exhibits symptoms associated with both stress incontinence and urge incontinence. Continuous urinary incontinence is the complaint of continuous leakage.

In another embodiment, a bladder disorder comprises an overactive bladder. Overactive bladder is increased urinary urgency, with or without urge urinary incontinence, usually with frequency and nocturia. The individual may report symptoms of urinary urgency (the sudden, intense desire to urinate immediately), urinary frequency (the need to urinate more times than is normal), enuresis (any involuntary loss of urine), polyuria, nocturia, and/or urinary incontinence. Thus, overactive bladder describes a bladder that contracts more often than it should, so that a person feels the need to urinate more frequently and/or urgently than necessary and is characterized by uncontrolled, frequent expulsion of urine from the bladder. An overactive bladder usually, but not always, causes urinary incontinence. Individuals with overactive bladder may go to the bathroom very often, e.g., every two hours during the day and night, and may even wet the bed. Often, a strong urge to void is experienced when only a small amount of urine is in the bladder. There may be reduced bladder capacity and incomplete emptying of urine. An overactive bladder can be caused by interruptions in the nerve pathways to the bladder occurring above the sacrum. For example, spastic bladder may be caused by an inability of the detrusor muscle of the bladder to inhibit emptying contractions until a reasonable amount of urine has accumulated. As such, overactive bladder is often associated with detrusor overactivity, a pattern of bladder muscle contraction observed during urodynamics. Overactive bladder can also be caused by urinary tract infection, outflow obstruction and stress incontinence. Sometimes no cause is found, and such idiopathic cases may be due to anxiety or aging. Symptoms include the need to urinate may times throughout the day and night, the sensation of having to urinate immediately, and/or the sudden leakage of urine from the bladder.

Diseases extrinsic to the bladder may also cause the symptoms of overactive bladder. In the male patient, the extrinsic disorder most often responsible for overactive bladder is bladder outlet obstruction (BOO). Disorders extrinsic to the bladder in the female patient include urethral diverticulum, retroverted uterus, pelvic prolapse (including cystocele), gravid uterus, and loss or reduction of estrogen. Disorders extrinsic to the bladder common to both men and woman include pelvic mass, physiologic nocturnal diuresis, and polyuria caused by factors such as excessive fluid intake, diuretic use, or diabetes. Neuromuscular disorders may also account for the overactive bladder. Neurogenic disorders resulting from nerve damage to sensory nerves can also cause overactive bladder, including, without limitation, Parkinson disease, multiple sclerosis, spina bifida, cervical stenosis, spinal cord injury, diabetic neuropathy, pelvic surgery, or invertebral disc herniation, hydrocephalus, stroke, spinal cord injuries and lesions of the spinal cord or brain. Bladder aging may also account for these symptoms. A patient history of pelvic trauma, pelvic radiation, or bladder, prostate, or urethral surgery should also be considered when seeking to determine the etiology of the overactive bladder.

In another embodiment, a bladder disorder comprises a detrusor dysfunction. A detrusor dysfunction, includes, without limitation, detrusor overactivity, detrusor instability, and detrusor-sphincter dyssynergia. One kind of detrusor dysfunction is detrusor overactivity or involuntary detrusor contractions (previously termed detrusor hyperreflexia). Detrusor overactivity involves increased involuntary contractions of the detrusor muscle during the filling phase which may be spontaneous or provoked resulting in uninhibitable bladder contractions. The muscle contraction patterns of detrusor overactivity include, without limitation, phasic detrusor overactivity and terminal detrusor overactivity. Detrusor overactivity can be either idiopathic in nature or they can be caused by non-neurogenic or neurogenic conditions. Symptoms of detrusor overactivity include, without limitation, uninhibitable bladder contractions, urinary urgency, urinary frequency, enuresis, polyuria, nocturia, and/or urinary incontinence. Another kind of detrusor dysfunction is detrusor instability. Detrusor instability involves uncontrolled involuntary contractions of the detrusor muscle resulting in uninhibitable bladder contractions irrespective of bladder capacity. Symptoms of detrusor instability include, without limitation, uninhibitable bladder contractions, urinary urgency, urinary frequency, enuresis, polyuria, nocturia, and/or urinary incontinence. Another kind of detrusor dysfunction is detrusor-sphincter dyssynergia (DSD). Detrusor-sphincter dyssynergia occurs when the contraction of the detrusor musculature is not coordinated with the relaxation of the sphincter thereby preventing the urethra from relaxing completely during voiding. Symptoms of detrusor-sphincter dyssynergia include, without limitation, urine flow interruption, raised detrusor pressure and/or urinary retention. DSD can be caused as a consequence of a neurological condition such as spinal injury or multiple sclerosis.

In another embodiment, a bladder disorder comprises a lower urinary tract dysfunction (LUTD). Lower urinary tract dysfunctions manifest three general types of symptoms: storage, voiding, and post-micturition symptoms. Storage symptoms are experienced during the storage phase of the bladder and include, without limitation, urinary urgency, urinary frequency, enuresis, polyuria, nocturia increased bladder sensation, decreased bladder sensation, absent bladder sensation, non-specific bladder sensation, and/or urinary incontinence. Voiding symptoms are experienced during the voiding phase. Symptoms include, without limitation, reduced urine flow, splitting or spraying of urine, intermittent urine flow, urinary hesitancy, strained effort to void urine, and/or terminal dribble of urine. Post-micturition symptoms are experienced immediately after micturition and include, without limitation, sensation of incomplete emptying and/or post-micturition dribble.

In another embodiment, a bladder disorder comprises an urinary retention. Urinary retention is the inability to pass urine from the bladder and may be either an acute or chronic condition. Normally, the reflex to urinate is triggered when the bladder fills to approximately 300-500 mL. The bladder is then emptied when the contraction of the bladder wall forces urine out through the urethra. The bladder, internal sphincters, and external sphincters may all be affected by disorders that create abnormalities in bladder function resulting in urinary retention. Urinary retention can result either from loss of bladder muscle contracting performance or loss of appropriate coordination between the bladder muscle and the urethral sphincter muscle. The inability to properly relax the urinary sphincter muscles causing difficulty in emptying the bladder, which can lead to urinary retention. Often, a strong urge to void is experienced when only a small amount of urine is in the bladder. In addition, there may be reduced bladder capacity and incomplete emptying of urine. Urinary retention may also be caused by difficulty in relaxing the urinary sphincter muscle because the sphincter may be spastic. Alternatively, the bladder neck may be hypertrophied. Other causes of urinary retention include interruptions in the nerve pathways to the bladder occurring above the sacrum. This nerve damage results in a loss of sensation and motor control and is often seen in stroke, Parkinson's disease, spina bifida, diabetes, pelvic surgery, or invertebral disc herniation, and most forms of spinal cord injuries. Sometimes no cause is found, and such idiopathic cases may be due to anxiety or aging. Urinary retention can also occur by a blockage to the flow of urine due to prostate enlargement or urinary tract stones. Another type of urinary retention disorder is stones, which block the urinary tract of an individual thereby causing stoppage of urine flow and/or infection. Either chronic or acute retention may lead to incontinence due to leakage of urine from an overfull bladder.

In another embodiment, a bladder disorder comprises an urinary hesitancy. Urinary hesitancy is difficulty starting or maintaining a urinary stream. This problem affects people of all ages and occurs in both sexes, but it is most common in older men with enlarged prostate glands. Urinary hesitancy usually comes on gradually. It sometimes goes unnoticed until urinary retention (complete inability to urinate) produces distention and discomfort in the bladder. Almost all older men have some degree of difficulty in starting urination, dribbling, or decreased force of the urinary stream. Urinary hesitancy can be caused by benign prostatic hyperplasia (enlarged prostate), urinary tract infection, especially if chronic and recurrent, prostatitis (inflammation or infection of the prostate gland), drugs (some cold remedies, some nasal decongestants, tricyclic antidepressants, and anticholinergics which may be used for incontinence), shy or bashful bladder syndrome in younger people (unable to urinate when another person is in the room), and neurological disorders.

In another embodiment, a bladder disorder comprises a polyuria. Polyuria is when an individual releases abnormally excessive volume of urine each day. An excessive volume of urination for an adult would be at least 2.5 liters of urine per day. Polyuria is a fairly common symptom, which is often noticed when you have to get up to use the bathroom at night.

In another embodiment, a bladder disorder comprises a nocturia. Nocturia is excessive urination at night, such as by waking up several times during the night to urinate. Normally, urine decreases in amount and become more concentrated at night. That means, most people can sleep 6 to 8 hours without having to urinate. But, persons with nocturia get up more than once during the night to urinate. Because of this, those who have excessive urination at night often have disrupted sleep cycles. Causes include benign prostatic hyperplasia, certain drugs including diuretics, cardiac glycosides, demeclocycline, lithium, methoxyflurane, phenytoin, propoxyphene, and excessive vitamin D, chronic or recurrent urinary tract infection, chronic renal failure, congestive heart failure, cystitis, diabetes, drinking too much fluid before bedtime, particularly coffee, caffeinated beverages, or alcohol, and obstructive sleep apnea and other sleeping disorders.

In another embodiment, a bladder disorder comprises a chronic urinary tract infection (recurrent infection). Chronic urinary tract infection (UTI) is a bacterial infection of the bladder or lower urinary tract (urethra) that lasts for a long time. Most urinary tract infections occur in the lower urinary tract, which includes the bladder and urethra. The condition occurs when the normally clean lower urinary tract is infected by bacteria and becomes inflamed. Urinary tract infections are very common. Most of the time, symptoms of a urinary tract infection disappear within 24-48 hours after treatment begins. However, if the condition occurs more than twice in 6 months, lasts longer than 2 weeks, or does not respond to usual treatment, it is considered chronic. The elderly are at increased risk for such infections because the bladder doesn't empty fully due to such conditions as benign prostatic hyperplasia, prostatitis, and urethral strictures. Other irritating symptoms may include painful urination (dysuria), which may be a result of a urinary tract infection (UTI) caused by urine being held too long in the bladder. UTI with fever is a sign of potential severe kidney infection (pyelonephritis) and is a more worrisome situation as it may result in permanent damage of the kidney(s). Another type of urinary tract infection is vesicoureteral reflux (VUR). Vesicoureteral reflux is an abnormal backup of urine from the bladder to the kidney(s) that occurs as a means of releasing high pressure within the bladder. A UTI is of particular concern as VUR may place the patient at significant risk for a severe kidney infection by transporting infected bladder urine directly to the kidney(s).

In another embodiment, a bladder disorder comprises a bladder disorder associated with a prostate disorder. The prostate is a partially glandular and partially fibromuscular organ of the male reproductive system that that produces the fluid that carries sperm during ejaculation. It surrounds the urethra, the tube through which urine passes out of the body. One type of prostate disorder is benign prostatic hyperplasia (BPH). During aging, the prostate tends to enlarge (hypertrophy) and this enlarged prostate is often called benign prostatic hyperplasia (BPH) or benign prostatic hypertrophy. Prostatic enlargement can lead to urethral obstruction and voiding dysfunction because the enlarged gland can press on the urethra. BPH is not cancer, and it does not raise your risk for prostate cancer. One type of prostate disorder is prostatitis. Prostatitis is an inflammation of the prostate gland. Prostatitis include acute and chronic bacterial prostatitis and inflammation not caused by bacterial infection (abacterial prostatitis). One type of prostate disorder is prostatodynia. Prostatodynia is a type of inflammation of the prostate not due to bacterial infection that may be caused by abnormal nerves or muscles in the region. Prostatodynia is typically a chronic, painful disease. The symptoms (including chills, fever, pain in the lower back and genital area, body aches, burning or painful urination, and the frequent and urgent need to urinate) characteristically go away and then come back without warning.

In another embodiment, a bladder disorder comprises a bladder disorder associated with a neurogenic dysfunction. For example, a bladder disorder disclosed herein may be associated with, e.g., Parkinson's Disease, multiple sclerosis, spina bifida, transverse myelitis, stroke, a spinal cord injury, a spasm reflex, or a neurologic lesion of the spinal cord or brain.

A composition or compound is administered to an individual. An individual comprises all mammals including a human being. Typically, any individual who is a candidate for a conventional bladder disorder treatment is a candidate for a bladder disorder treatment disclosed herein. Pre-operative evaluation typically includes routine history and physical examination in addition to thorough informed consent disclosing all relevant risks and benefits of the procedure.

The amount of a Clostridial toxin and/or a TEM disclosed herein used with the methods of treatment disclosed herein will typically be an effective amount. As used herein, the term “effective amount” is synonymous with “therapeutically effective amount”, “effective dose”, or “therapeutically effective dose” and when used in reference to treating a bladder disorder refers to the minimum dose of a Clostridial toxin and a TEM necessary to achieve the desired therapeutic effect and includes a dose sufficient to reduce a symptom associated with a bladder disorder. An effective amount refers to the total amount of a Clostridial toxin and/or TEM administered to an individual in one setting. As such, an effective amount of a Clostridial toxin and/or TEM does not refer to the amount administered per site. For example, an effective amount of a Clostridial toxin administered to an individual may be 10 U, whereas the amount of toxin administered per site may be 2 U, i.e., 2 U at five different sites. As another example, an effective amount of a Clostridial toxin administered to an individual may be 200 U, whereas the amount of toxin administered per site may be 5 U, i.e., 5 U at 40 different sites. The effectiveness of a Clostridial toxin and a TEM disclosed herein in treating a bladder disorder can be determined by observing an improvement in an individual based upon one or more clinical symptoms, and/or physiological indicators associated with the condition. An improvement in a bladder disorder also can be indicated by a reduced need for a concurrent therapy.

With reference to a combination therapy, in some embodiments an effective amount of a Clostridial toxin is one where in combination with a TEM the amount of a Clostridial toxin achieves the desired therapeutic effect, and is an amount that if administered alone, would also achieve a beneficial therapeutic effect (a normal or typical dose). For example, typically about 200 U of BOTOX® (Allergan, Inc., Irvine, Calif.), a BoNT/A, is administered by intramuscular injection into the detrusor muscle in order to treat overactive bladder. Thus, in some embodiments, the present specification discloses that a normal effective amount of a Clostridial toxin would be administered to treat a bladder disorder when such toxin is used in a combined therapy with a TEM. In aspects of this embodiment, an effective amount of BoNT/A administered to treat a bladder disorder when such toxin is used in a combined therapy with a TEM would be, e.g., at least 150 U, at least 175 U, at least 200 U, at least 225 U, at least 250 U, at least 275 U, or at least 300 U.

However, in other embodiments, with reference to a combination therapy comprising a Clostridial toxin and a TEM, an effective amount of a Clostridial toxin is one where in combination with a TEM the amount of a Clostridial toxin achieves the desired therapeutic effect, but such an amount administered on its own would be ineffective (a sub-optimal or non-optimal dose). Thus, in some embodiments, the present specification discloses that a suboptimal effective amount of a Clostridial toxin would be administered to treat a bladder disorder when such toxin is used in a combined therapy with a TEM. In aspects of this embodiment, an effective amount of BoNT/A administered to treat a bladder disorder when such toxin is used in a combined therapy with a TEM would be, e.g., less that 150 U, less than 125 U, less than 100 U, less than 75 U, less than 50 U, less than 25 U, less than 10 U, or less than 1 U.

The appropriate effective amount of a Clostridial toxin and a TEM to be administered to an individual for a particular bladder disorder can be determined by a person of ordinary skill in the art by taking into account factors, including, without limitation, the type of bladder disorder, the location of the bladder disorder, the cause of the bladder disorder, the severity of the bladder disorder, the degree of relief desired, the duration of relief desired, the particular TEM and/or Clostridial toxin used, the rate of excretion of the particular TEM and/or Clostridial toxin used, the pharmacodynamics of the particular TEM and/or Clostridial toxin used, the nature of the other compounds to be included in the composition, the particular route of administration, the particular characteristics, history and risk factors of the individual, such as, e.g., age, weight, general health and the like, or any combination thereof. Additionally, where repeated administration of a composition comprising a Clostridial toxin and/or a TEM is used, an effective amount of a Clostridial toxin and/or a TEM will further depend upon factors, including, without limitation, the frequency of administration, the half-life of the particular TEM and/or Clostridial toxin used, or any combination thereof. In is known by a person of ordinary skill in the art that an effective amount of a composition comprising a Clostridial toxin and/or TEM can be extrapolated from in vitro assays and in vivo administration studies using animal models prior to administration to humans.

In aspects of this embodiment, a therapeutically effective amount of a combination therapy comprising a TEM reduces a symptom associated with a bladder disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a combination therapy comprising a TEM reduces a symptom associated with a bladder disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a combination therapy comprising a TEM reduces a symptom associated with a bladder disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a TEM in a combination therapy is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a TEM in a combination therapy is generally in the range of about 1 fg to about 3.0 mg. In aspects of this embodiment, an effective amount of a TEM in a combination therapy can be, e.g., about 100 fg to about 3.0 mg, about 100 μg to about 3.0 mg, about 100 ng to about 3.0 mg, or about 100 μg to about 3.0 mg. In other aspects of this embodiment, an effective amount of a TEM in a combination therapy can be, e.g., about 100 fg to about 750 μg, about 100 μg to about 750 μg, about 100 ng to about 750 μg, or about 1 μg to about 750 μg. In yet other aspects of this embodiment, a therapeutically effective amount of a TEM in a combination therapy can be, e.g., at least 1 fg, at least 250 fg, at least 500 fg, at least 750 fg, at least 1 μg, at least 250 μg, at least 500 μg, at least 750 μg, at least 1 ng, at least 250 ng, at least 500 ng, at least 750 ng, at least 1 μg, at least 250 μg, at least 500 μg, at least 750 μg, or at least 1 mg. In still other aspects of this embodiment, a therapeutically effective amount of a composition comprising a TEM in a combination therapy can be, e.g., at most 1 fg, at most 250 fg, at most 500 fg, at most 750 fg, at most 1 μg, at most 250 μg, at most 500 μg, at most 750 μg, at most 1 ng, at most 250 ng, at most 500 ng, at most 750 ng, at most 1 μg, at least 250 μg, at most 500 μg, at most 750 μg, or at most 1 mg.

In yet other aspects of this embodiment, a therapeutically effective amount of a TEM in a combination therapy is generally in the range of about 0.00001 mg/kg to about 3.0 mg/kg. In aspects of this embodiment, an effective amount of a TEM in a combination therapy can be, e.g., about 0.0001 mg/kg to about 0.001 mg/kg, about 0.03 mg/kg to about 3.0 mg/kg, about 0.1 mg/kg to about 3.0 mg/kg, or about 0.3 mg/kg to about 3.0 mg/kg. In yet other aspects of this embodiment, a therapeutically effective amount of a TEM in a combination therapy can be, e.g., at least 0.00001 mg/kg, at least 0.0001 mg/kg, at least 0.001 mg/kg, at least 0.01 mg/kg, at least 0.1 mg/kg, or at least 1 mg/kg. In yet other aspects of this embodiment, a therapeutically effective amount of a TEM in a combination therapy can be, e.g., at most 0.00001 mg/kg, at most 0.0001 mg/kg, at most 0.001 mg/kg, at most 0.01 mg/kg, at most 0.1 mg/kg, or at most 1 mg/kg.

In aspects of this embodiment, a therapeutically effective amount of a combination therapy comprising a Clostridial toxin reduces a symptom associated with a bladder disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a combination therapy comprising a Clostridial toxin reduces a symptom associated with a bladder disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a combination therapy comprising a Clostridial toxin reduces a symptom associated with a bladder disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a Clostridial toxin in a combination therapy is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a Clostridial toxin in a combination therapy is generally in the range of about 1 fg to about 3.0 μg. In other aspects of this embodiment, a therapeutically effective amount of a Clostridial toxin in a combination therapy can be, e.g., at least 1.0 μg, at least 10 μg, at least 100 μg, at least 1.0 ng, at least 10 ng, at least 100 ng, at least 1.0 μg, at least 10 μg, at least 100 μg, or at least 1.0 mg. In still other aspects of this embodiment, a therapeutically effective amount of a Clostridial toxin in a combination therapy can be, e.g., at most 1.0 μg, at most 10 μg, at most 100 μg, at most 1.0 ng, at most 10 ng, at most 100 ng, at most 1.0 μg, at most 10 μg, at most 100 μg, or at most 1.0 mg. In still other aspects of this embodiment, a therapeutically effective amount of a Clostridial toxin in a combination therapy can be, e.g., about 1.0 μg to about 10 μg, about 10 μg to about 10 μg, about 100 μg to about 10 μg, about 1.0 ng to about 10 μg, about 10 ng to about 10 μg, or about 100 ng to about 10 μg. In still other aspects of this embodiment, a therapeutically effective amount of a Clostridial toxin in a combination therapy can be from, e.g., about 1.0 μg to about 1.0 μg, about 10 μg to about 1.0 μg, about 100 μg to about 1.0 μg, about 1.0 ng to about 1.0 μg, about 10 ng to about 1.0 μg, or about 100 ng to about 1.0 μg. In other aspects of this embodiment, a therapeutically effective amount of a Clostridial toxin in a combination therapy can be from, e.g., about 1.0 μg to about 100 ng, about 10 μg to about 100 ng, about 100 μg to about 100 ng, about 1.0 ng to about 100 ng, or about 10 ng to about 100 ng.

In yet other aspects of this embodiment, a therapeutically effective amount of a Clostridial toxin in a combination therapy is generally in the range of about 0.1 U to about 2500 U. In other aspects of this embodiment, a therapeutically effective amount of a Clostridial toxin in a combination therapy can be, e.g., at least 1.0 U, at least 10 U, at least 50 U, at least 100 U, at least 250 U, at least 500 U, at least 750 U, at least 1,000 U, at least 1,500 U, at least 2,000 U, or at least 2,500 U. In still other aspects of this embodiment, a therapeutically effective amount of a Clostridial toxin in a combination therapy can be, e.g., at most 1 U, at most 5 U, at most 10 U, at most 20 U, at most 25 U, at most 30 U, at most 40 U, at most 50 U, at most 60 U, at most 70 U, at most 75 U, at most 80 U, at most 90 U, at most 100 U, at most 125 U, at most 150 U, at most 175 U, at most 200 U, at most 250 U, at most 500 U, at most 750 U, at most 1,000 U, at most 1,500 U, at most 2,000 U, or at most 2,500 U. In still other aspects of this embodiment, a therapeutically effective amount of a Clostridial toxin in a combination therapy can be, e.g., about 1 U to about 2,000 U, about 10 U to about 2,000 U, about 50 U to about 2,000 U, about 100 U to about 2,000 U, about 500 U to about 2,000 U, about 1,000 U to about 2,000 U, about 1 U to about 1,000 U, about 10 U to about 1,000 U, about 50 U to about 1,000 U, about 100 U to about 1,000 U, about 500 U to about 1,000 U, about 1 U to about 500 U, about 10 U to about 500 U, about 50 U to about 500 U, about 100 U to about 500 U, about 1 U to about 200 U, about 10 U to about 200 U, about 25 U to about 200 U, about 50 U to about 200 U, about 75 U to about 200 U, about 100 U to about 200 U, about 150 U to about 200 U, about 1 U to about 150 U, about 10 U to about 150 U, about 25 U to about 150 U, about 50 U to about 150 U, about 75 U to about 150 U, about 100 U to about 150 U, about 1 U to about 125 U, about 10 U to about 125 U, about 25 U to about 125 U, about 50 U to about 125 U, about 75 U to about 125 U, about 100 U to about 125 U, about 1 U to about 100 U, about 10 U to about 100 U, about 25 U to about 100 U, about 50 U to about 100 U, about 75 U to about 100 U, about 0.1 U to about 1 U, about 0.1 U to about 5 U, about 0.1 U to about 10 U, about 0.1 U to about 15 U, about 0.1 U to about 20 U, or about 0.1 U to about 25 U.

In still other aspects of this embodiment, a therapeutically effective amount of a Clostridial toxin in a combination therapy is generally in the range of about 0.0001 U/kg to about 3,000 U/kg. In aspects of this embodiment, a therapeutically effective amount of a Clostridial toxin in a combination therapy can be, e.g., at least 0.001 U/kg, at least 0.01 U/kg, at least 0.1 U/kg, at least 1.0 U/kg, at least 10 U/kg, at least 100 U/kg, or at least 1000 U/kg. In other aspects of this embodiment, a therapeutically effective amount of a Clostridial toxin in a combination therapy can be, e.g., at most 0.001 U/kg, at most 0.01 U/kg, at most 0.1 U/kg, at most 1.0 U/kg, at most 10 U/kg, at most 100 U/kg, or at most 1000 U/kg. In yet other aspects of this embodiment, a therapeutically effective amount of a Clostridial toxin in a combination therapy can be between, e.g., about 0.001 U/kg to about 1 U/kg, about 0.01 U/kg to about 1 U/kg, about 0.1 U/kg to about 1 U/kg, about 0.001 U/kg to about 10 U/kg, about 0.01 U/kg to about 10 U/kg, about 0.1 U/kg to about 10 U/kg about 1 U/kg to about 10 U/kg, about 0.001 U/kg to about 100 U/kg, about 0.01 U/kg to about 100 U/kg, about 0.1 U/kg to about 100 U/kg, about 1 U/kg to about 100 U/kg, or about 10 U/kg to about 100 U/kg. As used herein, the term “unit” or “U” is refers to the LD₅₀ dose, which is defined as the amount of a Clostridial toxin disclosed herein that killed 50% of the mice injected with the Clostridial toxin.

In aspects of this embodiment, a therapeutically effective amount of a combined therapy comprising a Clostridial toxin and a TEM reduces a symptom associated with a bladder disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a combined therapy comprising a Clostridial toxin and a TEM reduces a symptom associated with a bladder disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a combined therapy comprising a Clostridial toxin and a TEM reduces a symptom associated with a bladder disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a combined therapy comprising a Clostridial toxin and a TEM is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a combined therapy comprising a Clostridial toxin and a TEM generally is in a Clostridial toxin: TEM molar ratio of about 1:1 to about 1:10,000. In other aspects of this embodiment, a therapeutically effective amount of a combined therapy comprising a Clostridial toxin and a TEM can be in a Clostridial toxin: TEM molar ratio of, e.g., about 1:1, about 1:2, about 1:5, about 1:10, about 1:25, about 1:50, about 1:75, about 1:100, about 1:200, about 1:300, about 1:400, about 1:500, about 1:600, about 1:700, about 1:800, about 1:900, about 1:1000, about 1:2000, about 1:3000, about 1:4000, about 1:5000, about 1:6000, about 1:7000, about 1:8000, about 1:9000, or about 1:10,000. In yet other aspects of this embodiment, a therapeutically effective amount of combined therapy comprising a Clostridial toxin and a TEM can be in a Clostridial toxin: TEM molar ratio of, e.g., at least 1:1, at least 1:2, at least 1:5, at least 1:10, at least 1:25, at least 1:50, at least 1:75, at least 1:100, at least 1:200, at least 1:300, at least 1:400, at least 1:500, at least 1:600, at least 1:700, at least 1:800, at least 1:900, at least 1:1000, at least 1:2000, at least 1:3000, at least 1:4000, at least 1:5000, at least 1:6000, at least 1:7000, at least 1:8000, at least 1:9000, or at least 1:10,000. In still other aspects of this embodiment, a therapeutically effective amount of a combined therapy comprising a Clostridial toxin and a TEM can be in a Clostridial toxin: TEM molar ratio of between, e.g., about 1:1 to about 1:10,000, about 1:10 to about 1:10,000, about 1:100 to about 1:10,000, about 1:500 to about 1:10,000, about 1:1000 to about 1:10,000, about 1:5000 to about 1:10,000, about 1:1 to about 1:1000, about 1:10 to about 1:1000, about 1:100 to about 1:1000, about 1:250 to about 1:1000, about 1:500 to about 1:1000, about 1:750 to about 1:1000, about 1:1 to about 1:500, about 1:10 to about 1:500, about 1:50 to about 1:500, about 1:100 to about 1:500, about 1:250 to about 1:500, about 1:1 to about 1:100, about 1:10 to about 1:100, about 1:25 to about 1:100, about 1:50 to about 1:100, or about 1:75 to about 1:100.

In yet other aspects of this embodiment, a therapeutically effective amount of a combined therapy comprising a Clostridial toxin and a TEM generally is in a range of about 0.01 U to about 200 U of Clostridial toxin and about 0.1 μg to about 1,000.0 μg of a TEM. In aspects of this embodiment, a therapeutically effective amount of a combined therapy comprising a Clostridial toxin and a TEM can be, e.g., about 0.1 U to about 150 U of a Clostridial toxin and about 10 μg to about 100 μg of a TEM, about 0.5 U to about 125 U of a Clostridial toxin and about 10 μg to about 100 μg of a TEM, about 0.5 U to about 100 U of a Clostridial toxin and about 10 μg to about 100 μg of a TEM, about 1 U to about 75 U of a Clostridial toxin and about 10 μg to about 100 μg of a TEM.

Dosing can be single dosage or cumulative (serial dosing), and can be readily determined by one skilled in the art. For instance, treatment of a bladder disorder may comprise a one-time administration of an effective dose of a composition disclosed herein. As a non-limiting example, an effective dose of a composition disclosed herein can be administered once to an individual, e.g., as a single injection or deposition at or near the site exhibiting a symptom of a bladder disorder. Alternatively, treatment of a bladder disorder may comprise multiple administrations of an effective dose of a composition disclosed herein carried out over a range of time periods, such as, e.g., daily, once every few days, weekly, monthly or yearly. As a non-limiting example, a composition disclosed herein can be administered once or twice yearly to an individual. The timing of administration can vary from individual to individual, depending upon such factors as the severity of an individual's symptoms. For example, an effective dose of a composition disclosed herein can be administered to an individual once a month for an indefinite period of time, or until the individual no longer requires therapy. A person of ordinary skill in the art will recognize that the condition of the individual can be monitored throughout the course of treatment and that the effective amount of a composition disclosed herein that is administered can be adjusted accordingly.

Various routes of administration can be useful for administering a therapeutic compound disclosed herein, according to a method of treating a bladder disorder as disclosed herein include both local and systemic administration. Local administration results in significantly more delivery of a composition to a specific location as compared to the entire body of the individual, whereas, systemic administration results in delivery of a composition to essentially the entire body of the individual. Routes of administration suitable for a method of treating a bladder disorder as disclosed herein also include both central and peripheral administration. Central administration results in delivery of a composition to essentially the central nervous system of an individual and includes, e.g., intrathecal administration, epidural administration as well as a cranial injection or implant. Peripheral administration results in delivery of a composition to essentially any area of an individual outside of the central nervous system and encompasses any route of administration other than direct administration to the spine or brain. The actual route of administration of a composition disclosed herein used can be determined by a person of ordinary skill in the art by taking into account factors, including, without limitation, the type of bladder disorder, the location of the bladder disorder, the cause of the bladder disorder, the severity of the bladder disorder, the degree of relief desired, the duration of relief desired, the particular Clostridial toxin and/or TEM used, the rate of excretion of the Clostridial toxin and/or TEM used, the pharmacodynamics of the Clostridial toxin and/or TEM used, the nature of the other compounds to be included in the composition, the particular route of administration, the particular characteristics, history and risk factors of the individual, such as, e.g., age, weight, general health and the like, or any combination thereof.

In an embodiment, a Clostridial toxin and/or TEM disclosed herein and/or composition thereof is administered by injection. In aspects of this embodiment, administration of a Clostridial toxin and/or TEM disclosed herein and/or composition thereof is by, e.g., intramuscular injection, intraorgan injection, subdermal injection, dermal injection, intracranical injection, spinal injection, or injection into any other body area for the effective administration of a composition disclosed herein. In aspects of this embodiment, injection of a Clostridial toxin and/or TEM disclosed herein and/or composition thereof is to a nerve or into the area surrounding a nerve. In another embodiment, a Clostridial toxin and/or TEM disclosed herein and/or composition thereof is administered by catheter. In aspects of this embodiment, administration of a Clostridial toxin and/or TEM disclosed herein and/or composition thereof is by, e.g., an epidural route of administration, an intravesical route of administration, intramuscular route of administration, subdermal route of administration, dermal route of administration, or intracranical route of administration. In aspects of this embodiment, administration of a Clostridial toxin and/or TEM disclosed herein and/or composition thereof is injected by a needle-based injection system like a syringe and needle system or a needleless injection system.

In an embodiment, a Clostridial toxin and/or TEM disclosed herein and/or composition thereof is topically administered. In aspects of this embodiment, a Clostridial toxin and/or TEM disclosed herein and/or composition thereof is formulated into a ointment, cream, salve, foam, gel or oil for topical administration.

A Clostridial toxin disclosed herein and a TEM disclosed herein can be administered into different treatment regions. In one embodiment, a Clostridial toxin disclosed herein or composition comprising a Clostridial toxin disclosed herein can be administered to a first treatment region and a TEM disclosed herein or a composition comprising a TEM disclosed herein can be administered into a second treatment region. In an aspect of this embodiment, a Clostridial toxin disclosed herein or composition comprising a Clostridial toxin disclosed herein can be administered to a first treatment region, but not into a second treatment region and a TEM disclosed herein or a composition comprising a TEM disclosed herein can be administered into a second treatment region, but not into a first treatment region.

A Clostridial toxin disclosed herein and a TEM disclosed herein can be administered into different treatment regions. In one embodiment, a Clostridial toxin disclosed herein or composition comprising a Clostridial toxin disclosed herein can be administered to a first treatment region of the urinary bladder and a TEM disclosed herein or a composition comprising a TEM disclosed herein can be administered into a second treatment region of the urinary bladder. In an aspect of this embodiment, a Clostridial toxin disclosed herein or composition comprising a Clostridial toxin disclosed herein can be administered to a first treatment region of the urinary bladder, but not into a second treatment region of the urinary bladder and a TEM disclosed herein or a composition comprising a TEM disclosed herein can be administered into a second treatment region of the urinary bladder, but not into a first treatment region of the urinary bladder.

In another aspect of this embodiment, a Clostridial toxin disclosed herein or composition comprising a Clostridial toxin disclosed herein can be administered into the left inferolateral bladder wall region, the right inferolateral bladder wall region, the posterior bladder wall region, and the dome region of a bladder, and a TEM disclosed herein or a composition comprising a TEM disclosed herein can be administered into the trigone region. In yet another aspect of this embodiment, a Clostridial toxin or composition comprising a Clostridial toxin disclosed herein can be administered into the left inferolateral bladder wall region, the right inferolateral bladder wall region, the posterior bladder wall region, and the dome region of a bladder, but not the trigone region of the bladder treated by a TEM or composition thereof, and a TEM or a composition comprising a TEM disclosed herein can be administered into the trigone region, but not in the bladder wall regions treated by the Clostridial toxin or composition thereof.

In another aspect of this embodiment, a Clostridial toxin disclosed herein or composition comprising a Clostridial toxin disclosed herein can be administered in. e.g., about 15 to about 20 sites, about 15 to about 25 sites, about 20 to about 25 sites, about 15 to about 30 sites, or about 20 to about 30 sites evenly spaced in the regions comprising the left inferolateral bladder wall, the right inferolateral bladder wall, the posterior bladder wall, and the dome of a bladder, and a TEM disclosed herein or a composition comprising a TEM disclosed herein can be administered in. e.g., about 1 to about 5 sites, about 2 to about 6 sites, about 3 to about 7 sites, about 4 to about 8 sites evenly spaced in the region comprising the trigone. In yet another aspect of this embodiment, a Clostridial toxin or composition comprising a Clostridial toxin disclosed herein can be administered in. e.g., about 15 to about 20 sites, about 15 to about 25 sites, about 20 to about 25 sites, about 15 to about 30 sites, or about 20 to about 30 sites evenly spaced in the regions comprising the left inferolateral bladder wall, the right inferolateral bladder wall, the posterior bladder wall, and the dome of a bladder, but not the trigone region being treated by a TEM or composition thereof, and a TEM or a composition comprising a TEM disclosed herein can be administered in. e.g., about 1 to about 5 sites, about 2 to about 6 sites, about 3 to about 7 sites, about 4 to about 8 sites evenly spaced in the region comprising the trigone, but not in the bladder wall regions being treated by the Clostridial toxin or composition thereof.

A composition disclosed herein as disclosed herein can also be administered to an individual in combination with other therapeutic compounds to increase the overall therapeutic effect of the treatment. The use of multiple compounds to treat an indication can increase the beneficial effects while reducing the presence of side effects.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, compositions, methods or uses of treating a bladder disorder.

### Example 1

### Treatment of Urinary Incontinence

A female complains of the inability to control the passage of urine. A physician diagnosis the patient with urinary incontinence having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates there is improvement of her ability to control the passage of urine. At one and three month check-ups, the woman indicates that she continues to have increased control over her ability to pass urine. This reduction in an urinary incontinence symptom indicates successful treatment with a combination therapy as disclosed herein.

A female complains of the inability to control the passage of urine, and leakage occurs especially when she coughs, sneezes, laughs or exercises. A physician diagnosis the patient with stress urinary incontinence having a neurological component involving abnormal motor neuron activity as well as abnormal sensory, sympathetic and/or parasympathetic neuron activity. The woman is treated by injecting urethroscopically a composition comprising a Clostridial toxin disclosed herein at multiple different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates there is improvement of her ability to control the passage of urine, especially when she coughs, sneezes, laughs or exercises. At one and three month check-ups, the woman indicates that she continues to have increased control over her ability to pass urine. This reduction in a stress urinary incontinence symptom indicates successful treatment with a combination therapy as disclosed herein.

A male complains of the inability to control the passage of urine, experiencing a sudden need to urinate. A physician diagnosis the patient with urge urinary incontinence having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates there is improvement of his ability to control the passage of urine because of a reduced sudden need to urinate. At one and three month check-ups, the man indicates that he continues to have increased control over his ability to pass urine. This reduction in an urge urinary incontinence symptom indicates successful treatment with a combination therapy as disclosed herein.

A male complains of the inability to control the passage of urine because of leakage that occurs. A physician diagnosis the patient with overflow urinary incontinence having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates there is improvement of his ability to control the passage of urine because of reduced leakage. At one and three month check-ups, the man indicates that he continues to have increased control over his ability to pass urine. This reduction in an overflow urinary incontinence symptom indicates successful treatment with a combination therapy as disclosed herein.

### Example 2

### Treatment of Overactive Bladder

A male complains of increased urinary urgency. A physician diagnosis the patient with overactive bladder having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that he has a reduced urgency to urinate. At one and three month check-ups, the man indicates that he continues to have a reduced urgency to urinate. This reduction in an overactive bladder symptom indicates successful treatment with a combination therapy as disclosed herein.

A female complains of having to wake up several times during the night to urinate. A physician determines that this is nocturia and diagnosis the patient with overactive bladder having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that she has a reduced need to wake up several times during the night to urinate. At one and three month check-ups, the woman indicates that she continues to have a reduced need to wake up several times during the night to urinate. This reduction in an overactive bladder symptom indicates successful treatment with a combination therapy as disclosed herein.

A female complains of having to urinate several times a day. A physician determines that this is polyuria and diagnosis the patient with overactive bladder having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that she has a reduced need to urinate during the day. At one and three month check-ups, the woman indicates that she continues to have a reduced need urinate during the day. This reduction in an overactive bladder symptom indicates successful treatment with a combination therapy as disclosed herein.

A male complains of the inability to control the passage of urine because of a sudden need to urinate. A physician determines that this is urge incontinence and diagnosis the patient with overactive bladder having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that he has a reduced urgency to urinate. At one and three month check-ups, the man indicates that he continues to have a reduced urgency to urinate. This reduction in an overactive bladder symptom indicates successful treatment with a combination therapy as disclosed herein.

### Example 3

### Treatment of Detrusor Dysfunction

A female complains of uncontrollable bladder contractions. A physician determines that this is uninhibitable bladder contractions and diagnosis the patient with a detrusor dysfunction having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in uncontrollable bladder contractions. At one and three month check-ups, the woman indicates that she continues to have a reduction in uncontrollable bladder contractions. This reduction in a detrusor dysfunction symptom indicates successful treatment with a combination therapy as disclosed herein.

In an alternative scenario, the physician determines that this is uninhibitable bladder contractions and diagnosis the patient with detrusor overactivity having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in uncontrollable bladder contractions. At one and three month check-ups, the woman indicates that she continues to have a reduction in uncontrollable bladder contractions. This reduction in a detrusor overactivity symptom indicates successful treatment with a combination therapy as disclosed herein.

In another alternative scenario, the physician determines that this is uninhibitable bladder contractions and diagnosis the patient with detrusor instability having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in uncontrollable bladder contractions. At one and three month check-ups, the woman indicates that she continues to have a reduction in uncontrollable bladder contractions. This reduction in a detrusor instability symptom indicates successful treatment with a combination therapy as disclosed herein.

A female complains of an urgency to urinate. A physician determines that this is urinary urgency and diagnosis the patient with a detrusor dysfunction having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in the urgency to urinate. At one and three month check-ups, the woman indicates that she continues to have a reduction in the urgency to urinate. This reduction in a detrusor dysfunction symptom indicates successful treatment with a combination therapy as disclosed herein.

In an alternative scenario, the physician determines that this is urinary urgency and diagnosis the patient with detrusor overactivity having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in the urgency to urinate. At one and three month check-ups, the woman indicates that she continues to have a reduction in the urgency to urinate. This reduction in a detrusor overactivity symptom indicates successful treatment with a combination therapy as disclosed herein.

In another alternative scenario, the physician determines that this is urinary urgency and diagnosis the patient with detrusor instability having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in the urgency to urinate. At one and three month check-ups, the woman indicates that she continues to have a reduction in the urgency to urinate. This reduction in a detrusor instability symptom indicates successful treatment with a combination therapy as disclosed herein.

A male complains of having to urinate all the time. A physician determines that this is urinary frequency and diagnosis the patient with a detrusor dysfunction having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the need to urinate all the time. At one and three month check-ups, the man indicates that he continues to have a reduction in the need to urinate all the time. This reduction in a detrusor dysfunction symptom indicates successful treatment with a combination therapy as disclosed herein.

In an alternative scenario, the physician determines that this is urinary frequency and diagnosis the patient with detrusor overactivity having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the need to urinate all the time. At one and three month check-ups, the man indicates that he continues to have a reduction in the need to urinate all the time. This reduction in a detrusor overactivity symptom indicates successful treatment with a combination therapy as disclosed herein.

In another alternative scenario, the physician determines that this is urinary frequency and diagnosis the patient with detrusor instability having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the need to urinate all the time. At one and three month check-ups, the man indicates that he continues to have a reduction in the need to urinate all the time. This reduction in a detrusor instability symptom indicates successful treatment with a combination therapy as disclosed herein.

A male complains of the involuntary loss of urine. A physician determines that this is enuresis and diagnosis the patient with a detrusor dysfunction having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the involuntary loss of urine. At one and three month check-ups, the man indicates that he continues to have a reduction in the involuntary loss of urine. This reduction in a detrusor dysfunction symptom indicates successful treatment with a combination therapy as disclosed herein.

In an alternative scenario, the physician determines that this is enuresis and diagnosis the patient with detrusor overactivity having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the involuntary loss of urine. At one and three month check-ups, the man indicates that he continues to have a reduction in the involuntary loss of urine. This reduction in a detrusor overactivity symptom indicates successful treatment with a combination therapy as disclosed herein.

In another alternative scenario, the physician determines that this is enuresis and diagnosis the patient with detrusor instability having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the involuntary loss of urine. At one and three month check-ups, the man indicates that he continues to have a reduction in the involuntary loss of urine. This reduction in a detrusor instability symptom indicates successful treatment with a combination therapy as disclosed herein.

A male complains of having to wake up several times during the night to urinate. A physician determines that this is nocturia and diagnosis the patient with a detrusor dysfunction having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in need to wake up several times during the night to urinate. At one and three month check-ups, the man indicates that he continues to have a reduction in need to wake up several times during the night to urinate. This reduction in a detrusor dysfunction symptom indicates successful treatment with a combination therapy as disclosed herein.

In an alternative scenario, the physician determines that this is nocturia and diagnosis the patient with detrusor overactivity having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in need to wake up several times during the night to urinate. At one and three month check-ups, the man indicates that he continues to have a reduction in need to wake up several times during the night to urinate. This reduction in a detrusor overactivity symptom indicates successful treatment with a combination therapy as disclosed herein.

In another alternative scenario, the physician determines that this is nocturia and diagnosis the patient with detrusor instability having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in need to wake up several times during the night to urinate. At one and three month check-ups, the man indicates that he continues to have a reduction in need to wake up several times during the night to urinate. This reduction in a detrusor instability symptom indicates successful treatment with a combination therapy as disclosed herein.

A female complains of having to urinate several times a day. A physician determines that this is polyuria and diagnosis the patient with a detrusor dysfunction having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in the need to urinate several times a day. At one and three month check-ups, the woman indicates that she continues to have a reduction in the need to urinate several times a day. This reduction in a detrusor dysfunction symptom indicates successful treatment with a combination therapy as disclosed herein.

In an alternative scenario, the physician determines that this is polyuria and diagnosis the patient with detrusor overactivity having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in the need to urinate several times a day. At one and three month check-ups, the woman indicates that she continues to have a reduction in the need to urinate several times a day. This reduction in a detrusor overactivity symptom indicates successful treatment with a combination therapy as disclosed herein.

In another alternative scenario, the physician determines that this is polyuria and diagnosis the patient with detrusor instability having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in the need to urinate several times a day. At one and three month check-ups, the woman indicates that she continues to have a reduction in the need to urinate several times a day. This reduction in a detrusor instability symptom indicates successful treatment with a combination therapy as disclosed herein.

A female complains of the inability to control the passage of urine. A physician determines that this is urinary incontinence and diagnosis the patient with a detrusor dysfunction having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from the treatment, and the woman indicates there is improvement of her ability to control the passage of urine. At one and three month check-ups, the woman indicates that she continues to have an improved ability to control the passage of urine since the treatment. This reduction in a detrusor dysfunction symptom indicates successful treatment with a combination therapy as disclosed herein.

In an alternative scenario, the physician determines that this is urinary incontinence and diagnosis the patient with detrusor overactivity having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from the treatment, and the woman indicates there is improvement of her ability to control the passage of urine. At one and three month check-ups, the woman indicates that she continues to have an improved ability to control the passage of urine since the treatment. This reduction in a detrusor overactivity symptom indicates successful treatment with a combination therapy as disclosed herein.

In another alternative scenario, the physician determines that this is urinary incontinence and diagnosis the patient with detrusor instability having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from the treatment, and the woman indicates there is improvement of her ability to control the passage of urine. At one and three month check-ups, the woman indicates that she continues to have an improved ability to control the passage of urine since the treatment. This reduction in a detrusor instability symptom indicates successful treatment with a combination therapy as disclosed herein.

A female complains of an interruption of urine flow when she urinates. A physician diagnosis the patient with a detrusor dysfunction having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in urine flow interruption. At one and three month check-ups, the woman indicates that she continues to have a reduced urine flow interruption since the treatment. This reduction in a detrusor dysfunction symptom indicates successful treatment with a combination therapy as disclosed herein.

In an alternative scenario, the physician diagnosis the patient with a detrusor-sphincter dyssynergia having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in urine flow interruption. At one and three month check-ups, the woman indicates that she continues to have a reduced urine flow interruption since the treatment. This reduction in a detrusor-sphincter dyssynergia symptom indicates successful treatment with a combination therapy as disclosed herein.

A male complains of increased bladder pressure. A physician determines that this is raised detrusor pressure and diagnosis the patient with a detrusor dysfunction having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in bladder pressure. At one and three month check-ups, the man indicates that he continues to have a reduced bladder pressure since the treatment. This reduction in a detrusor dysfunction symptom indicates successful treatment with a combination therapy as disclosed herein.

In an alternative scenario, the physician determines that this is raised detrusor pressure and diagnosis the patient with a detrusor-sphincter dyssynergia having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in bladder pressure. At one and three month check-ups, the man indicates that he continues to have a reduced bladder pressure since the treatment. This reduction in a detrusor-sphincter dyssynergia symptom indicates successful treatment with a combination therapy as disclosed herein.

A male complains of the inability to urinate. A physician determines that this is urinary retention and diagnosis the patient with a detrusor dysfunction having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that he has regained the ability to urinate. At one and three month check-ups, the man indicates that he continues to have the ability to urinate. This reduction in a detrusor dysfunction symptom indicates successful treatment with a combination therapy as disclosed herein.

In an alternative scenario, the physician determines that this is urinary retention and diagnosis the patient with a detrusor-sphincter dyssynergia having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that he has regained the ability to urinate. At one and three month check-ups, the man indicates that he continues to have the ability to urinate. This reduction in a detrusor-sphincter dyssynergia symptom indicates successful treatment with a combination therapy as disclosed herein.

### Example 4

### Treatment of Lower Urinary Tract Dysfunction

A male complains of the need to urinate suddenly. A physician determines that this is a urine storage problem and diagnosis the patient with a lower urinary tract dysfunction having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the sudden need to urinate. At one and three month check-ups, the man indicates that he still experiences a reduced need to urinate. This reduction in a lower urinary tract dysfunction indicates successful treatment with a combination therapy as disclosed herein. In similar scenarios the patient could have complained of other storage symptoms of lower urinary tract dysfunction such as, e.g., urinary frequency, enuresis, polyuria, nocturia increased bladder sensation, decreased bladder sensation, absent bladder sensation, non-specific bladder sensation, and/or urinary incontinence. In each case, after diagnosis of lower urinary tract dysfunction, a physician would treat the patient as indicated above and there would be a reduction in the lower urinary tract dysfunction storage symptom.

A male complains of having difficulty urinating and having to strain in order to urinate. A physician determines that this is a urine voiding problem and diagnosis the patient with a lower urinary tract dysfunction having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that it is easier to urinate and he does not have to strain as much in order to urinate. At one and three month check-ups, the man indicates that he still experiences an easier time to urinate. This reduction in a lower urinary tract dysfunction indicates successful treatment with a combination therapy as disclosed herein. In similar scenarios the patient could have complained of other voiding symptoms of lower urinary tract dysfunction such as, e.g., reduced urine flow, splitting or spraying of urine, intermittent urine flow, urinary hesitancy, and/or terminal dribble of urine. In each case, after diagnosis of lower urinary tract dysfunction, a physician would treat the patient as indicated above and there would be a reduction in the lower urinary tract dysfunction voiding symptom.

A male complains of urine dribbling after he finishes urinating. A physician determines that this is a urine post-micturition problem and diagnosis the patient with a lower urinary tract dysfunction having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in urine dribbling after he finishes urinating. At one and three month check-ups, the man indicates that he still experiences reduced dribbling after he finishes urinating. This reduction in a lower urinary tract dysfunction indicates successful treatment with a combination therapy as disclosed herein. In similar scenarios the patient could have complained of other post-micturition symptoms of lower urinary tract dysfunction such as, e.g., sensation of incomplete emptying. In each case, after diagnosis of lower urinary tract dysfunction, a physician would treat the patient as indicated above and there would be a reduction in the lower urinary tract dysfunction post-micturition symptom.

### Example 5

### Treatment of Urinary Retention

A female complains that she cannot urinate. A physician diagnosis the patient with urinary retention having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that she has regained the ability to urinate. At one and three month check-ups, the woman indicates that she still continues to have control over her ability to urinate. This reduction in a urinary retention symptom indicates successful treatment with a combination therapy as disclosed herein.

### Example 6

### Treatment of Urinary Hesitancy

A male complains that he has difficulty starting and/or maintaining his ability to urinate. A physician diagnosis the patient with urinary hesitancy having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that he has less difficulty in starting and/or maintaining his ability to urinate. At one and three month check-ups, the man indicates that he still experiences less difficulty in starting and/or maintaining his ability to urinate. This reduction in a urinary hesitancy symptom indicates successful treatment with a combination therapy as disclosed herein.

### Example 7

### Treatment of Polyuria

A male complains that he has to urinate all the time during the day. A physician diagnosis the patient with polyuria having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that does not have to urinate as many times during the day as before the treatment. At one and three month check-ups, the man still indicates that does not have to urinate as many times during the day as before the treatment. This reduction in a polyuria symptom indicates successful treatment with a combination therapy as disclosed herein.

### Example 8

### Treatment of Nocturia

A female complains that she has to wake up several times during the night in order to urinate. A physician diagnosis the patient with nocturia having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that she does not have to get up as many times during the night to urinate as she did before the treatment. At one and three month check-ups, the woman still indicates that she does not have to get up as many times during the night to urinate as she did before the treatment. This reduction in a nocturia symptom indicates successful treatment with a combination therapy as disclosed herein.

### Example 9

### Treatment of Chronic Urinary Tract Infection

A female complains that she has urinary tract infections all the time. A physician determines that the chronic urinary tract infections is a bacterial and diagnosis the patient with a bladder disorder having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the physician indicates that she does not have a urinary tract infection. At one and three month check-ups, the woman indicates that she has not had a urinary tract infection since the treatment. This reduction in a urinary tract infection symptom indicates successful treatment with a combination therapy as disclosed herein.

A female complains that she has urinary tract infections all the time. A physician determines that the chronic urinary tract infection is due to vesicoureteral reflux and diagnosis the patient with a bladder disorder having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the physician determines that the abnormal backup of urine from the bladder to the kidneys is reduced in the patient. At one and three month check-ups, the woman indicates that she has not had a urinary tract infection since the treatment. This reduction in a urinary tract infection symptom indicates successful treatment with a combination therapy as disclosed herein.

### Example 10

### Treatment of a Bladder Disorder Associated with a Neurogenic Dysfunction

A female diagnosed with Parkinson's Disease complains about having a sudden need to urinate. A physician determines that this urinary urgency is due to her Parkinson's Disease and diagnosis the patient with a bladder disorder associated with a neurogenic dysfunction having a neurological component involving abnormal motor neuron activity as well as abnormal sensory, sympathetic and/or parasympathetic neuron activity. The woman is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in the sudden need to urinate. At one and three month check-ups, the woman indicates that she continues to experience a reduced sudden need to urinate. This reduction in a urogenital disorder symptom associated with a neurogenic dysfunction indicates successful treatment with a combination therapy as disclosed herein.

A female diagnosed with multiple sclerosis complains about having a need to urinate all the time. A physician determines that this urinary frequency is due to her multiple sclerosis and diagnosis the patient with a bladder disorder associated with a neurogenic dysfunction having a neurological component involving abnormal motor neuron activity as well as abnormal sensory, sympathetic and/or parasympathetic neuron activity. The woman is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the woman is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, or lower pelvic muscles. The patient's condition is monitored and after about 1-3 days from treatment, and the woman indicates that there is a reduction in the need to urinate all the time. At one and three month check-ups, the woman indicates that she still experiences a reduced need to urinate all the time. This reduction in a urogenital disorder symptom associated with a neurogenic dysfunction indicates successful treatment with a combination therapy as disclosed herein.

A male diagnosed with spina bifida complains about the inability to control the passage of urine. A physician determines that this urinary incontinence is due to his spina bifida and diagnosis the patient with a bladder disorder associated with a neurogenic dysfunction having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the boy indicates that he has an increased ability to control the passage or urine. At one and three month check-ups, the boy indicates that he still experiences an increased ability to control the passage or urine. This reduction in a urogenital disorder symptom associated with a neurogenic dysfunction indicates successful treatment with a combination therapy as disclosed herein.

A male who experienced a stroke complains about not being able to urinate. A physician determines that this urinary retention is due to his stroke and diagnosis the patient with a bladder disorder associated with a neurogenic dysfunction having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that he can urinate. At one and three month check-ups, the man indicates that he continues to experience the ability to urinate. This reduction in a urogenital disorder symptom associated with a neurogenic dysfunction indicates successful treatment with a combination therapy as disclosed herein.

A male suffering from a spinal cord injury resulting from a car accident complains about the inability to control the passage of urine. A physician determines that this urinary incontinence is due to his spinal cord injury and diagnosis the patient with a bladder disorder associated with a neurogenic dysfunction having a neurological component involving abnormal motor neuron activity as well as abnormal sensory, sympathetic and/or parasympathetic neuron activity. The man is treated by injecting urethroscopically a composition comprising a Clostridial toxin disclosed herein at 20 different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that he has an increased ability to control the passage or urine. At one and three month check-ups, the man indicates that he still experiences an increased ability to control the passage or urine. This reduction in a urogenital disorder symptom associated with a neurogenic dysfunction indicates successful treatment with a combination therapy as disclosed herein.

A male who has cancerous lesion in his brain complains about having a need to urinate all the time. A physician determines that this urinary frequency is due to his lesion and diagnosis the patient with a bladder disorder associated with a neurogenic dysfunction having a neurological component involving abnormal motor neuron activity as well as abnormal sensory, sympathetic and/or parasympathetic neuron activity. The man is treated by administering urethroscopically a composition comprising a Clostridial toxin disclosed herein at a plurality of different sites in the bladder muscles located in the left and right inferolateral bladder wall regions, the posterior bladder wall region, and the dome region. In the same procedure, the man is treated by injecting urethroscopically a composition comprising a TEM disclosed herein at three sites in the bladder muscles located in the trigone region. The administration may be with a needle, a needleless device, or other needleless delivery system. Depending on the location of abnormal sensory, sympathetic and/or parasympathetic neuron activity, the TEM may also be administered into the muscles of the left and right inferolateral bladder wall regions, the posterior bladder wall region, the bladder neck including the internal urethral sphincter, the bladder dome, and/or other areas surrounding the bladder, such as, e.g., the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the need to urinate all the time. At one and three month check-ups, the man indicates that he still experiences a reduced need to urinate all the time. This reduction in a urogenital disorder symptom associated with a neurogenic dysfunction indicates successful treatment with a combination therapy as disclosed herein.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating a bladder disorder in an individual, the method comprising the steps of:
administering to the individual in need thereof a therapeutically effective amount of a first composition comprising a botulinum neurotoxin to a first treatment region;
administering a therapeutically effective amount of a second composition including a targeted exocytosis modulator (TEM) to a second treatment region; wherein the TEM comprises a targeting domain, a Clostridial toxin translocation domain, a Clostridial toxin enzymatic domain, and an exogenous protease cleavage site, wherein the targeting domain is a sensory neuron targeting domain, a sympathetic neuron targeting domain, or a parasympathetic neuron targeting domain, wherein the first treatment region includes a lateral bladder wall region, a bladder dome region, or both,
wherein the second treatment region includes a trigone region, a bladder base region, or both, and, wherein administration of the first and second compositions reduces a symptom of the bladder disorder, thereby treating the individual.

2. The method of claim 1, wherein the first treatment region includes a lateral bladder wall region, a bladder dome region, or both, but excludes both a trigone region and a bladder base region.

3. The method of claim 1, wherein the bladder disorder is an urinary incontinence, an overactive bladder, a detrusor dysfunction, a lower urinary tract dysfunction, an urinary retention, an urinary hesitancy, a polyuria, a nocturia, a chronic urinary tract infection, a bladder disorder associated with a prostate disorder, or a baldder disorder associated with a neurogenic dysfunction.

4. A kit for the treatment of a bladder disorder in an individual in need thereof comprising a first composition including a botulinum neurotoxin, and a second composition including a TEM, wherein the botulinum neurotoxin is administered to a lateral bladder wall region, a bladder dome region, or both; and the TEM is administered to a trigone region, a bladder base region, or both.
